SEQUENCE LISTING (17 sequences)

SEQ ID NO: 1 - protein, 183 aa, Artificial Sequence
  “Amino acid sequence of the core antigen (HBcAg)”
  Amino acid sequence of the core antigen (HBcAg)
MDIDPYKEFGASVELLSFLPSDFFPSIRDLLDTASALYREALESPEHCSPHHTALRQAILCWGELMNLATWVGSNLEDPASRELVVSYVNVNMGLKIRQLLWFHISCLTFGRETVLEYLVSFGVWIRTPPAYRPPNAPILSTLPETTVVRRRGRSPRRRTPSPRRRRSQSPRRRRSQSRESQC

SEQ ID NO: 2 - DNA, 552 bp, Artificial Sequence
  “nucleotide sequence encoding the hepatitis B virus core antigen (HBcAg) ”
  nucleotide sequence encoding the hepatitis B virus core antigen (HBcAg)
atggacattgacccgtataaagaatttggagcttctgtggagttactctcttttttgccttctgacttctttccttctattcgagatctcctcgacaccgcctctgctctgtatcgggaggccttagagtctccggaacattgttcacctcaccatacggcactcaggcaagctattctgtgttggggtgagttaatgaatctagccacctgggtgggaagtaatttggaagatccagcatccagggaattagtagtcagctatgtcaacgttaatatgggcctaaaaatcagacaactattgtggtttcacatttcctgtcttacttttgggagagaaactgttcttgaatatttggtgtcttttggagtgtggattcgcactcctcccgcatatagaccgccaaatgcccctatcttatcaacacttccggaaactactgttgttagacgaagaggcaggtcccctagaagaagaactccctcgcctcgcagacgaaggtctcaatcgccgcgtcgcagaagatctcaatctcgggaatctcaatgttag

SEQ ID NO: 3 - DNA, 552 bp, Artificial Sequence
  “HBV Core codon optimized sequence ”
  HBV Core codon optimized sequence
atggacatcgacccttacaaggagttcggcgccagcgtggagctcctgagcttcctgcccagcgacttctttcccagcatcagagacctgctggacaccgccagcgccctgtacagggaagccctggagagccccgagcactgtagccctcaccacaccgccctcagacaggccatcctgtgctggggcgagctgatgaacctggccacctgggtgggaagcaacctggaagaccccgcctccagggagctggtggtgtcctacgtgaacgtgaacatgggcctgaagatcaggcagctgctgtggttccacatcagctgcctgaccttcggcagagaaaccgtgctggagtacctggtgagcttcggcgtctggatcagaacccctcctgcctacagaccccctaacgcccccatcctgtccaccctgcccgagaccacagtggtgaggaggagaggcaggagccccagaaggaggacccctagccccaggaggaggagaagccagtcccccaggagaagaaggtcccagtccagggagagccagtgctga

SEQ ID NO: 4 - protein, 119 aa, Artificial Sequence
  “amino acid sequence of the PreS1 antigen (PreS1)”
  amino acid sequence of the PreS1 antigen (PreS1)
MGGWSSKPRKGMGTNLSVPNPLGFFPDHQLDPAFKANSENPDWDLNPHKDNWPDANKVGVGAFGPGFTPPHGGLLGWSPQAQGLLTTVPAAPPPASTNRQLGRQPTPLSPPLRDTHPQA

SEQ ID NO: 5 - DNA, 360 bp, Artificial Sequence
  “HBV genotype B preS1 coding sequence ”
  HBV genotype B preS1 coding sequence
atgggaggttggtcttccaaacctcgaaaaggcatggggacaaatctttctgtccccaatcccctgggattcttccccgatcatcagttggaccctgcattcaaagccaactcagaaaatccagattgggacctcaacccgcacaaggacaactggccggacgccaacaaggtgggagtgggagcattcgggccagggttcacccctccccatgggggactgttggggtggagccctcaggctcagggcctactcacaactgtgccagcagctcctcctcctgcctccaccaatcggcagttaggaaggcagcctactcccttatctccacctctaagggacactcatcctcaggcctga

SEQ ID NO: 6 - DNA, 360 bp, Artificial Sequence
  “HBV genotype B preS1 codon optimized sequence (adw-preS1) ”
  HBV genotype B preS1 codon optimized sequence (adw-preS1)
atgggcggctggagcagcaagcccagaaagggcatgggcaccaacctgagcgtgcccaaccccctgggcttcttccccgaccaccagctggaccccgccttcaaggccaacagcgagaaccccgactgggacctgaacccccacaaggacaactggcctgacgccaacaaggtgggcgtgggagctttcggccctggcttcacccctccccatggaggactgctgggctggagccctcaggctcagggactgctgaccacagtgcccgctgctcctcctcctgccagcaccaacaggcagctgggcagacagcccacacccctgagccctcctctgagagacacccacccccaggcctga

SEQ ID NO: 7 - protein, 119 aa, Artificial Sequence
  “HBV Type C preS1 amino acid sequence ”
  HBV Type C preS1 amino acid sequence
MGGWSSKPRKGMGTNLSVPNPLGFFPDHQLDPAFGANSNNPDWDFNPNKDHWPEANQVGAGAFGPGFTPPHGGLLGWSPQAQGILTTVPVAPPPASTNRQSGRQPTPISPPLRDSHPQA

SEQ ID NO: 8 - DNA, 360 bp, Artificial Sequence
  “HBV genotype B preS1 coding sequence ”
  HBV genotype B preS1 coding sequence
atgggaggttggtcttccaaacctcgaaaaggcatggggacgaatctttctgttcccaatcctctgggattctttcccgatcaccagttggaccctgcgttcggagccaactcaaacaatccagattgggacttcaaccccaacaaggatcactggccagaggcaaatcaggtaggagcgggagcattcgggccagggttcaccccaccacacggcggtcttttggggtggagccctcaggctcaaggcatattgacaacagtgccagtagcacctcctcctgcctccaccaatcggcagtcagggagacagcctactcccatctctccacctctaagagacagtcatcctcaggcctga

SEQ ID NO: 9 - DNA, 360 bp, Artificial Sequence
  “HBV genotype C preS1 codon optimized sequence (adr-preS1)”
  HBV genotype C preS1 codon optimized sequence (adr-preS1)
atgggcggatggtcctccaagcctagaaagggcatgggcaccaatctgtccgtgcccaaccccctgggctttttccccgatcaccagttagatcctgccttcggcgccaacagcaacaaccctgactgggacttcaaccccaacaaggatcactggcccgaggccaatcaagtgggcgctggagctttcggccctggcttcacacctccccatggaggactgctgggctggtcccctcaggcccagggaattctcacaacagtgcccgtggcccctcctcccgctagcaccaacaggcagagcggcaggcagcccacacctatcagcccccctctgagagattcccacccccaggcctga

SEQ ID NO: 10 - protein, 400 aa, Artificial Sequence
  “HBV Type B Large S amino acid sequence ”
  HBV Type B Large S amino acid sequence
MGGWSSKPRKGMGTNLSVPNPLGFFPDHQLDPAFKANSENPDWDLNPHKDNWPDANKVGVGAFGPGFTPPHGGLLGWSPQAQGLLTTVPAAPPPASTNRQLGRQPTPLSPPLRDTHPQAMQWNSTTFHQTLQDPRVRALYFPAGGSSSGTVSPAQNTVSAISSILSKTGDPVPNMENIASGLLGPLLVLQAGFFLLTKILTIPQSLDSWWTSLNFLGGTPVCLGQNSQSQISSHSPTCCPPICPGYRWMYLRRFIIFLCILLLCLIFLLVLLDYQGMLPVCPLIPGSSTTSTGPCKTCTTPAQGTSMFPSCCCTKPTDGNCTCIPIPSSWAFAKFLWEWASVRFSWLSLLVPFVQWFVGLSPTVWLSVIWMIWFWGPSLYNILSPFMPLLPIFFCLWVYI

SEQ ID NO: 11 - DNA, 1203 bp, Artificial Sequence
  “HBV genotype B large S coding sequence ”
  HBV genotype B large S coding sequence
atgggaggttggtcttccaaacctcgaaaaggcatggggacaaatctttctgtccccaatcccctgggattcttccccgatcatcagttggaccctgcattcaaagccaactcagaaaatccagattgggacctcaacccgcacaaggacaactggccggacgccaacaaggtgggagtgggagcattcgggccagggttcacccctccccatgggggactgttggggtggagccctcaggctcagggcctactcacaactgtgccagcagctcctcctcctgcctccaccaatcggcagttaggaaggcagcctactcccttatctccacctctaagggacactcatcctcaggccatgcagtggaactccaccactttccaccaaactcttcaagatcccagagtcagggccctgtactttcctgctggtggctccagttcaggaacagtgagccctgctcaaaatactgtctctgccatatcgtcaatcttatcgaaaactggggaccctgtaccgaacatggagaacatcgcatcaggactcctaggacccctgctcgtgttacaggcggggtttttcttgttgacaaaaatcctcacaataccacagagtctagactcgtggtggacttctctcaattttctagggggaacacccgtgtgtcttggccaaaattcgcagtcccaaatctccagtcactcaccaacctgttgtcctccaatttgtcctggttatcgctggatgtatctgcggcgttttatcatattcctctgcatcctgctgctatgcctcatcttcttgttggttcttctggactatcaaggtatgttgcccgtttgtcctctaattccaggatcatcaacaaccagcaccggaccatgcaaaacctgcacgactcctgctcaaggaacctctatgtttccctcatgttgctgtacaaaacctacggacggaaactgcacctgtattcccatcccatcatcttgggctttcgcaaaattcctatgggagtgggcctcagtccgtttctcttggctcagtttactagtgccatttgttcagtggttcgtagggctttcccccactgtctggctttcagttatatggatgatttggttttgggggccaagtctgtacaacatcttgagtccctttatgccgctgttaccaattttcttttgtctttgggtatacatttaa

SEQ ID NO: 12 - DNA, 1203 bp, Artificial Sequence
  “HBV genotype B Large S codon optimized sequence (adw-LargeS) ”
  HBV genotype B Large S codon optimized sequence (adw-LargeS)
atgggaggctggtcctccaaacccaggaagggcatgggcacaaacctgtccgtgcccaaccctctgggctttttccccgaccaccagctggaccccgccttcaaggctaacagcgagaaccccgactgggacctgaatccccacaaggacaattggcccgatgccaataaggtgggcgtgggcgccttcggccctggatttacacccccccatggaggactgctgggatggtcccctcaggcccaaggcctgctgaccacagtgcccgctgctcctccccctgcttccaccaatagacagctcggcagacagcccacacccctgtcccctcctctgagggacacccatccccaggccatgcagtggaatagcaccaccttccaccagacactgcaggaccccagggtgagagccctgtacttccccgccggaggttctagcagcggaacagtgagccccgcccagaacacagtgtccgccatcagcagcattctgtccaagacaggcgaccccgtgcccaacatggagaacatcgccagcggactcctgggacctctcctggtgctgcaggccggcttcttcctgctgaccaagatcctgaccatcccccagagcctggattcctggtggaccagcctgaactttctgggaggcacccccgtgtgcctgggccagaatagccagagccagatctcctcccacagccctacctgctgcccccctatctgccctggatacaggtggatgtacctgaggaggttcatcatcttcctgtgcattctgctgctgtgcctcatctttctgctggtgctgctggattaccagggcatgctgcctgtgtgtcccctgatccccggcagcagcaccacaagcaccggcccctgtaagacctgtaccacccccgcccagggaacctccatgttcccttcctgctgctgcaccaagcccaccgacggcaactgtacatgcatccccattcccagcagctgggcctttgctaaattcctgtgggagtgggcctccgtgagattcagctggctgtccctgctggtgcctttcgtgcagtggttcgtgggactgtcccccacagtgtggctgtccgtgatctggatgatctggttctggggccccagcctgtacaacatcctgagccctttcatgcccctgctgcccatcttcttttgcctctgggtgtacatttga

SEQ ID NO: 13 - protein, 400 aa, Artificial Sequence
  “HBV Type C Large S amino acid sequence ”
  HBV Type C Large S amino acid sequence
MGGWSSKPRKGMGTNLSVPNPLGFFPDHQLDPAFGANSNNPDWDFNPNKDHWPEANQVGAGAFGPGFTPPHGGLLGWSPQAQGILTTVPVAPPPASTNRQSGRQPTPISPPLRDSHPQAIQWNSTTFHQALLDPRVRGLYFPAGGSSSGTVNPVPTTASPTSSIFSRTGDPAPNMENTTSGFLGPLLVLQAGFFLLTRILTIPQSLDSWWTSLNFLGGAPTCPGQNSQSPTSNHSPTSCPPICPGYRWMCLRRFIIFLFILLLCLIFLLVLLDYQGMLPVCPLLPGTSTTSTGPCKTCTIPAQGTSMFPSCCCTKPSDGNCTCIPIPSSWAFARFLWEWASVRFSWLSLLVPFVQWFVGLSPTVWLSVIWMMWYWGPSLYNILSPFLPLLPIFFCLWVYI

SEQ ID NO: 14 - DNA, 1203 bp, Artificial Sequence
  “HBV genotype C large S coding sequence ”
  HBV genotype C large S coding sequence
atgggaggttggtcttccaaacctcgaaaaggcatggggacgaatctttctgttcccaatcctctgggattctttcccgatcaccagttggaccctgcgttcggagccaactcaaacaatccagattgggacttcaaccccaacaaggatcactggccagaggcaaatcaggtaggagcgggagcattcgggccagggttcaccccaccacacggcggtcttttggggtggagccctcaggctcaaggcatattgacaacagtgccagtagcacctcctcctgcctccaccaatcggcagtcagggagacagcctactcccatctctccacctctaagagacagtcatcctcaggccatacagtggaattccacaacattccaccaagctctgctagaccccagagtgaggggcctatactttcctgctggtggctccagttccggaacagtaaaccctgttccgactactgcctcacccacatcgtcaatcttctcgaggactggggaccctgcaccgaacatggagaacacaacatcaggattcctaggacccctgctcgtgttacaggcggggtttttcttgttgacaagaatcctcacaataccacagagtctagactcgtggtggacttctctcaattttctagggggagcacccacgtgtcctggccaaaattcgcagtccccaacctccaatcactcaccaacctcttgtcctccaatttgtcctggctatcgctggatgtgtctgcggcgttttatcatattcctcttcatcctgctgctatgcctcatcttcttgttggttcttctggactaccaaggtatgttgcccgtttgtcctctacttccaggaacatcaactaccagcacaggaccatgcaagacctgcacgattcctgctcaaggaacctctatgtttccctcttgttgctgtacaaaaccttcggacggaaactgcacttgtattcccatcccatcatcctgggctttcgcaagattcctatgggagtgggcctcagtccgtttctcctggctcagtttactagtgccatttgttcagtggttcgtagggctttcccccactgtttggctttcagttatatggatgatgtggtattgggggccaagcctgtacaacatcttgagtccctttttacctctattaccaattttcttttgtctttgggtatacatttga

SEQ ID NO: 15 - DNA, 1203 bp, Artificial Sequence
  “HBV genotype C Large S codon optimized sequence (adr-LargeS) ”
  HBV genotype C Large S codon optimized sequence (adr-LargeS)
atgggcggatggtcctccaagcctagaaagggcatgggcaccaatctgtccgtgcccaaccccctgggctttttccccgatcaccagttagatcctgccttcggcgccaacagcaacaaccctgactgggacttcaaccccaacaaggatcactggcccgaggccaatcaagtgggcgctggagctttcggccctggcttcacacctccccatggaggactgctgggctggtcccctcaggcccagggaattctcacaacagtgcccgtggcccctcctcccgctagcaccaacaggcagagcggcaggcagcccacacctatcagcccccctctgagagattcccacccccaggccatccagtggaatagcaccaccttccaccaagccctgctcgaccctagggtgaggggcctgtactttcccgctggcggcagctccagcggcacagtgaatcccgtgcccacaaccgcctcccctacctcctccattttcagcagaaccggcgaccccgcccccaacatggagaacacaacctccggctttctcggccctctgctggtgctgcaggccggcttcttcctgctgacccgtattttaaccatcccccagagcctggactcctggtggacctccctgaacttcctgggaggagcccctacctgtcccggacagaactcccagtcccctaccagcaatcactcccccaccagctgccctcctatctgccccggctacaggtggatgtgtctgaggaggttcatcatcttcctgttcatcctgctgctgtgcctcattttcctgctggtgctgctggactaccagggcatgctgcccgtgtgtcctctgctgcccggcacaagcaccacaagcaccggcccctgcaagacctgtaccatccccgcccagggcacctccatgtttccctcctgctgctgcaccaagccctccgatggcaactgcacctgcatccctatcccctcctcctgggccttcgccagattcctgtgggaatgggcttccgtgaggttctcctggctgtccctgctggtgccctttgtgcagtggtttgtgggcctgagccctacagtgtggctgtccgtgatctggatgatgtggtattggggcccctccctgtacaacatcctgagccccttcctgcccctgctgcccatcttcttctgcctgtgggtgtacatctga

SEQ ID NO: 16 - DNA, 717 bp, Artificial Sequence
  “GFP coding sequence ”
  GFP coding sequence
atgagtaaaggagaagaacttttcactggagttgtcccaattcttgttgaattagatggtgatgttaatgggcacaaattttctgtcagtggagagggtgaaggtgatgcaacatacggaaaacttacccttaaatttatttgcactactggaaaactacctgttccatggccaacacttgtcactactttcggttatggtgttcaatgctttgcgagatacccagatcatatgaaacagcatgactttttcaagagtgccatgcctgaaggttatgtacaggaaagaactatatttttcaaagatgacgggaactacaagacacgtgctgaagtcaagtttgaaggtgatacccttgttaatagaatcgagttaaaaggtattgattttaaagaagatggaaacattcttggacacaaattggaatacaactataactcacacaatgtatacatcatggcagacaaacaaaagaatggaatcaaagttaacttcaaaattagacacaacattgaagatggaagcgttcaactagcagaccattatcaacaaaatactccaattggcgatggccctgtccttttaccagacaaccattacctgtccacacaatctgccctttcgaaagatcccaacgaaaagagagaccacatggtccttcttgagtttgtaacagctgctgggattacacatggcatggatgaactatacaaataa

SEQ ID NO: 17 - DNA, 3182 bp, Hepatitis B virus
  “AAV8-1.3HBV ”
  AAV8-1.3HBV
ccacaacctttcaccaaactctgcaagatcccagagtgagaggcctgtatttccctgctggtggctccagttcaggagcagtaaaccctgttccgactactgcctctcccttatcgtcaatcttctcgaggattggggaccctgcgctgaacatggagaacatcacatcaggattcctaggaccccttctcgtgttacaggcggggtttttcttgttgacaagaatcctcacaataccgcagagtctagactcgtggtggacttctctcaattttctagggggaactaccgtgtgtcttggccaaaattcgcagtccccaacctccaatcactcaccaacctcctgtcctccaacttgtcctggttatcgctggatgtgtctgcggcgttttatcatcttcctcttcatcctgctgctatgcctcatcttcttgttggttcttctggactatcaaggtatgttgcccgtttgtcctctaattccaggatcctcaaccaccagcacgggaccatgccgaacctgcatgactactgctcaaggaacctctatgtatccctcctgttgctgtaccaaaccttcggacggaaattgcacctgtattcccatcccatcatcctgggctttcggaaaattcctatgggagtgggcctcagcccgtttctcctggctcagtttactagtgccatttgttcagtggttcgtagggctttcccccactgtttggctttcagttatatggatgatgtggtattgggggccaagtctgtacagcatcttgagtccctttttaccgctgttaccaattttcttttgtctttgggtatacatttaaaccctaacaaaacaaagagatggggttactctctgaattttatgggttatgtcattggaagttatgggtccttgccacaagaacacatcatacaaaaaatcaaagaatgttttagaaaacttcctattaacaggcctattgattggaaagtatgtcaacgaattgtgggtcttttgggttttgctgccccatttacacaatgtggttatcctgcgttaatgcccttgtatgcatgtattcaatctaagcaggctttcactttctcgccaacttacaaggcctttctgtgtaaacaatacctgaacctttaccccgttgcccggcaacggccaggtctgtgccaagtgtttgctgacgcaacccccactggctggggcttggtcatgggccatcagcgcgtgcgtggaaccttttcggctcctctgccgatccatactgcggaactcctagccgcttgttttgctcgcagcaggtctggagcaaacattatcgggactgataactctgttgtcctctcccgcaaatatacatcgtatccatggctgctaggctgtgctgccaactggatcctgcgcgggacgtcctttgtttacgtcccgtcggcgctgaatcctgcggacgacccttctcggggtcgcttgggactctctcgtccccttctccgtctgccgttccgaccgaccacggggcgcacctctctttacgcggactccccgtctgtgccttctcatctgccggaccgtgtgcacttcgcttcacctctgcacgtcgcatggagaccaccgtgaacgcccaccgaatgttgcccaaggtcttacataagaggactcttggactctctgcaatgtcaacgaccgaccttgaggcatacttcaaagactgtttgtttaaagactgggaggagttgggggaggagattagattaaaggtctttgtactaggaggctgtaggcataaattggtctgcgcaccagcaccatgcaactttttcacctctgcctaatcatctcttgttcatgtcctactgttcaagcctccaagctgtgccttgggtggctttggggcatggacatcgacccttataaagaatttggagctactgtggagttactctcgtttttgccttctgacttctttccttcagtacgagatcttctagataccgcctcagctctgtatcgggaagccttagagtctcctgagcattgttcacctcaccatactgcactcaggcaagcaattctttgctggggggaactaatgactctagctacctgggtgggtgttaatttggaagatccagcatctagagacctagtagtcagttatgtcaacactaatatgggcctaaagttcaggcaactcttgtggtttcacatttcttgtctcacttttggaagagaaaccgttatagagtatttggtgtctttcggagtgtggattcgcactcctccagcttatagaccaccaaatgcccctatcctatcaacacttccggaaactactgttgttagacgacgaggcaggtcccctagaagaagaactccctcgcctcgcagacgaaggtctcaatcgccgcgtcgcagaagatctcaatctcgggaacctcaatgttagtattccttggactcataaggtggggaactttactggtctttattcttctactgtacctgtctttaatcctcattggaaaacaccatcttttcctaatatacatttacaccaagacattatcaaaaaatgtgaacagtttgtaggcccacttacagttaatgagaaaagaagattgcaattgattatgcctgctaggttttatccaaaggttaccaaatatttaccattggataagggtattaaaccttattatccagaacatctagttaatcattacttccaaactagacactatttacacactctatggaaggcgggtatattatataagagagaaacaacacatagcgcctcattttgtgggtcaccatattcttgggaacaagatctacagcatggggcagaatctttccaccagcaatcctctgggattctttcccgaccaccagttggatccagccttcagagcaaacacagcaaatccagattgggacttcaatcccaacaaggacacctggccagacgccaacaaggtaggagctggagcattcgggctgggtttcaccccaccgcacggaggccttttggggtggagccctcaggctcagggcatactacaaactttgccagcaaatccgcctcctgcctccaccaatcgccagacaggaaggcagcctaccccgctgtctccacctttgagaaacactcatcctcaggccatgcagtggaatt

(19) United States
(12) Patent Application Publication
(10) Pub. No.: US 2025/0073332 A1
(43) Pub. Date: Mar. 6, 2025

(54) LENTIVIRAL VECTOR, LENTIVIRAL PARTICLE FOR TREATING HEPATITIS B AND ITS PREPARATION METHOD AND APPLICATION THEREOF

(71) Applicants: THERAVECTYS, Paris Cedex 15 (FR); SHANGHAI JINWEI BIOTECHNOLOGY CO., LTD., Shanghai (CN); INSTITUT PASTEUR, Paris Cedex 15 (FR)

(72) Inventors: Pierre Alain CHARNEAU, Paris Cedex 15 (FR); Maryline BOURGINE, Paris Cedex 15 (FR); Chao QIU, Shanghai (CN); Ye TIAN, Paris Cedex 15 (FR); Benjamin VESIN, Paris Cedex 15 (FR)

(21) Appl. No.: 18/285,553

(22) PCT Filed: Apr. 7, 2022
(86) PCT No.: PCT/EP2022/059281

§ 371 Date: Oct. 4, 2023

(30) Foreign Application Priority Data
Apr. 7, 2021 (CN) 202110374234.2

(51) Int. Cl. A61K 39/29; A61K 39/00; A61P 31/20; A61P 37/04; C07K 14/005; C12N 15/86

(52) U.S. Cl. CPC A61K 39/292; A61P 31/20; A61P 37/04; C07K 14/005; C12N 15/86; A61K 2039/5256; A61K 2039/53; C12N 2730/10134; C12N 2740/15034; C12N 2740/15043; C12N 2740/15052

(57) ABSTRACT

The invention discloses a lentiviral vector and lentiviral vector particles for treating hepatitis B infection. The lentiviral vector and particles of the invention contain a nucleotide sequence coding an hepatitis B virus antigen. The large S antigen of hepatitis B virus was in particular selected as candidate and can be applied in pharmaceutical compositions or vaccines for treating and/or preventing hepatitis B virus infection or treating and/or preventing diseases caused by hepatitis B virus infection, which has excellent therapeutic and preventive effects in subjects in need thereof.

### TECHNICAL FIELD

This application relates to the field of biomedicine, and specifically to lentiviral vectors and lentiviral particles for treating and/or preventing hepatitis B, and a preparation method and application thereof.

### TECHNICAL BACKGROUND

Hepatitis B virus (HBV) infection is one of the serious public health problems worldwide. HBV infection is an important cause of chronic hepatitis B, liver cirrhosis and hepatocellular carcinoma. Commonly used drugs for clinical treatment of chronic HBV infection mainly include nucleoside analogs and interferons. Nucleoside analogs cannot completely remove HBV DNA in liver cells, and long-term use can easily lead to the emergence of drug-resistant mutant strains and rebound after drug withdrawal.

After a course of interferon treatment (usually 48 weeks), the incidence of HBeAg seroconversion is only 33%, and the HBsAg conversion rate is less than 10%. Interferon monotherapy has a negative conversion rate of 3%-7% for HBsAg, which is only slightly higher than that of nucleoside analogues, and the large side effects of interferon also limit its application.

Currently, the widely used hepatitis B protein vaccine induces humoral immunity and produces protective antibodies to achieve the purpose of prevention. A large number of studies have found that protective antibodies can only eliminate extracellular virus particles, but cannot eliminate intracellular viruses, and it is difficult to treat infected patients. After antiviral treatment, the incidence of hepatocellular carcinoma in HBsAg-positive people is significantly higher than that of HBsAg-negative people. HBsAg-negative conversion is related to improvement of liver function, improvement of histopathology, and improvement of long-term prognosis. It is currently recommended by the latest domestic and foreign chronic hepatitis B prevention guidelines. The ideal treatment goal is functional cure, so HBsAg negative, that is, functional cure or clinical cure, is the main goal of the current chronic hepatitis B treatment. The art still needs drugs that can be used to treat and/or prevent HBV infection or treat and/or prevent diseases caused by HBV.

The HBV sequence inserted in the lentiviral vector in the patent CN109923212A is the surface antigen of genotype A and C, Pol, HBx, MHC I and MHC II epitope sequence, and the surface B cell epitope of genotype A and C. Combined with VLP, and the results shown are only immunogenic results, the therapeutic effect is unknown. The antigen sequence disclosed by CN1209340A is aligned to the HBV genotype A sequence, which is mainly distributed in Europe and Central Africa.

### CONTENT OF THE INVENTION

The present invention provides a lentiviral vector for the treatment of hepatitis B. The lentiviral vector comprises a nucleotide sequence encoding at least one hepatitis B virus antigen, and the hepatitis B virus antigen is selected from the group consisting of core antigen (HBcAg), PreS1 antigen (PreS1) and large S antigen (LargeS). Preferably, the encoded hepatitis B virus antigen is the large S antigen (LargeS).

The amino acid sequence of the core antigen (HBcAg) may be the sequence set forth as SEQ ID NO: 1; and/or the amino acid sequence of the PreS1 antigen (PreS1) may be the sequence set forth as SEQ ID NO: 4 or as SEQ ID NO: 7; and/or the amino acid sequence of the large S antigen (LargeS) may be the sequence set forth as SEQ ID NO: 10 or as SEQ ID NO: 13.

The coding nucleotide sequence of the core antigen (HBcAg) may be the sequence set forth as SEQ ID NO: 3; and/or the coding nucleotide sequence of the PreS1 antigen (PreS1) may be the sequence set forth as SEQ ID NO: 6 or as SEQ ID NO: 9; and/or the coding nucleotide sequence of the large S antigen (LargeS) may be the sequence set forth as SEQ ID NO: 12 or as SEQ ID NO: 15.

The present invention also provides a method for preparing lentiviral particles for treating hepatitis B, characterized in that the method comprises the steps of:
a) co-transfecting host cells with the lentiviral vector according to the invention, a packaging vector expressing Gag, Rev and/or Pol protein, and an envelope vector expressing an envelope protein, or transfecting the lentiviral vector according to the invention into a host cell capable of expressing an envelope protein and one or more of Gag, Rev, and Pol proteins;
b) culturing the transfected host cell to package the lentiviral vector into lentiviral vector particles; and
c) harvesting the lentiviral vector particles produced in step b).

The present invention also provides a preparation of lentiviral particles for the treatment of hepatitis B. The preparation of lentiviral particles comprises the lentiviral vector according to the invention or the lentiviral particles are prepared by the preparation method according to the invention.

The present invention also provides a lentiviral vector according to the invention or a preparation of lentiviral particles according to the invention for the treatment and/or prevention of hepatitis B virus infection in a subject in need thereof, or for treatment and/or prevention of diseases caused by hepatitis B virus infection in a subject in need thereof. The lentiviral vector according to the invention or preparation of lentiviral particles according to the invention may in particular be implemented in a pharmaceutical composition, such as in a vaccine, in particular with a pharmaceutically acceptable carrier.

A subject according to the invention may in particular be a mammal, such as a human. The present invention also provides a pharmaceutical composition for treating and/or preventing hepatitis B virus infection or treating and/or preventing a disease caused by hepatitis B virus infection in a subject in need thereof, the pharmaceutical composition comprising a lentiviral vector according to the invention or a preparation of lentiviral particles according to the invention, and a pharmaceutically acceptable carrier.

The beneficial effects of the present invention include:

The immunogen of the present invention can cover most of the epidemic strain sequences in China, and the non-integrating lentiviral vector according to the invention can optimize antigen presentation and break immune tolerance under the premise of ensuring its safety. The animal experiment data included herein proves its good immunogenicity and can induce a strong immune response in wild mice. In the mouse model of chronic HBV infection, significant therapeutic effects have also been confirmed, including the elimination of HBV antigens and viral DNA, the conversion of antibodies to positive, and the ability to activate a strong T cell immune response, which has important clinical transformation potential.

### FIGURE LEGENDS

FIGS. 1a and 1b are a plasmid maps;

FIG. 2 shows the experimental timeline of the tests presented herein;

FIG. 3 shows the detection of HBsAg in mice. FIGS. 3A-3E show AAV-HBV-infected mice intramuscularly injected with lentivirus JW27, JW28, JW27+JW28, JW29 and JW30 (abscissa: time (weeks); ordinate: HBsAg (IU/mL));

FIG. 4 is the detection of HBV-DNA in mice. FIGS. 4A-4E show AAV-HBV-infected mice intramuscularly injected with lentivirus JW27, JW28, JW27+JW28, JW29 and JW30 (abscissa: time (weeks); ordinate: HBV-DNA Log 10 (IU/mL));

FIGS. 5A to 5E are graphs showing the results of using the mouse IFN-γ ELISPOT assay to analyze the induced T cell responses, in which FIGS. 5A-5E are the JW27, JW28, JW27+JW28, JW29 and JW30 groups in sequence (abscissa: from left to right: HBsAg Pool; PreS1-GT B Pool; PreS1-GT C Pool; PreS2 Pool and Core Pool; ordinate: SFC/10⁶ splenocytes);

FIG. 6 shows the results of HBcAg immunohistochemistry in liver tissues of the various tested mice in the following order from left to right and top to bottom: JW27-259, JW27-273, JW27-269, JW28-225, JW28-264, JW28-266, JW27+JW28-234, JW27+JW28-236, JW27+JW28-201, JW29-211; JW29-249, JW29-268, JW30-226, JW30-227 and JW30-260.

### SPECIFIC IMPLEMENTATION

The present invention will be further illustrated and described below in conjunction with embodiments, but the described embodiments are only a part of the embodiments of the present invention, rather than all the embodiments. Based on the present invention and the embodiments, all other inventions and embodiments obtained by those of ordinary skill in this field without creative work shall fall within the protection scope of the present invention.

The materials and reagents used in the following examples can be obtained from commercial sources unless otherwise specified.

Unless otherwise indicated or defined, all the terms used have their usual meanings in this field, which will be understood by those skilled in this field. Reference is made, for example, to standard manuals such as Sambrook et al., “Molecular Cloning: A Laboratory Manual; 2000”; Lewin, “Genes VIII”; and Roitt et al., “Immunology” (8th edition), as well as the general existing Technology. In addition, unless otherwise specified, all methods, steps, techniques and operations that are not specifically detailed can be and have been performed in a manner known per se, which will be understood by those skilled in the field of the present invention. It is also referred to, for example, the standard manual, the above-mentioned general prior art and other references cited therein.

As used herein, the term “and/or” encompasses all combinations of items connected by the term, and should be treated as if each combination has been individually listed herein. For example, “A and/or B” encompasses “A”, “A and B”, and “B”. For example, “A, B and/or C” encompasses “A”, “B”, “C”, “A and B”, “A and C”, “B and C”, and “A and B and C”.

When the terms “contains” or “comprises” are used herein to describe the sequence of a protein or nucleic acid, the protein or nucleic acid may be composed of the sequence, or may have additional amino acids or cores at one or both ends of the protein or nucleic acid, but still has the activity described in the present invention. In addition, it is clear to those skilled in this field that the methionine encoded by the start codon at the N-terminus of the polypeptide will be retained under certain practical conditions (for example, when expressed in a specific expression system), but does not substantially affect the function of the polypeptide. Therefore, when describing a specific amino acid sequence of a polypeptide in the specification and claims of the text, although it may not contain the methionine encoded by the start codon at the N-terminus, it also covers those containing the methionine. The coding nucleotide sequence, correspondingly, may also contain a start codon; and vice versa.

“Polynucleotide”, “nucleic acid sequence”, “nucleotide sequence” or “nucleic acid fragment” are used interchangeably and are single-stranded or double-stranded RNA or DNA polymers, optionally containing synthetic, non-natural Or changed nucleotide bases. Nucleotides are referred to by their single letter names as follows: “A” is adenosine or deoxyadenosine (respectively for RNA or DNA), “C” is cytidine or deoxycytidine, and “G” is guanosine or Deoxyguanosine, “U” means uridine, “T” means deoxythymidine, “R” means purine (A or G), “Y” means pyrimidine (C or T), “K” means G or T, “H” means A or C or T, “D” means A, T or G, “I” means inosine, and “N” means any nucleotide.

“Polypeptide”, “peptide”, and “protein” are used interchangeably in the present invention and refer to a polymer of amino acid residues. The term applies to amino acid polymers in which one or more amino acid residues are corresponding artificial chemical analogs of naturally occurring amino acids, as well as to naturally occurring amino acid polymers. The terms “polypeptide”, “peptide”, “amino acid sequence” and “protein” may also include modified forms, including but not limited to glycosylation, lipid linkage, sulfation, gamma carboxylation of glutamic acid residues, hydroxyl And ADP-ribosylation.

“Regulatory sequence” and “regulatory element” are used interchangeably and refer to 20 the upstream (5′ non-coding sequence), middle, or downstream (3′ non-coding sequence) of the coding sequence, and affect the transcription, RNA processing, or processing of the related coding sequence, or stability of the translated nucleotide sequence. Regulatory sequences may include, but are not limited to, promoters, translation leader sequences, introns, and polyadenylation recognition sequences.

As used herein, the term “operably linked” refers to the connection of regulatory elements (for example, but not limited to, promoter sequences, transcription termination sequences, etc.) to nucleic acid sequences (for example, coding sequences or open reading frames) such that the transcription of the nucleotide sequence is controlled and regulated by the transcription control element. Techniques for operably linking regulatory element regions to nucleic acid molecules are known.

The terms “treatment” or “treating” refer to administering an active agent with the purpose to cure, heal, alleviate, relieve, alter, remedy, ameliorate, improve, or affect the considered condition (e.g., a disease), the symptoms of the condition, or to prevent or delay of the onset of the symptoms, complications, biochemical indicia of a disease, or otherwise arrest or inhibit further development of the disease, condition, or disorder in a statistically significant manner.

The terms “prevent”, “preventing” and “prevention” refer to reducing the likelihood of acquiring a considered disease and/or one or all of its symptoms.

### 1. Lentiviral Vector Expressing Hepatitis B Virus (HBV) Antigen, Lentiviral Particle and Preparation Method Thereof

The present invention provides a lentiviral vector comprising a nucleotide sequence encoding a hepatitis B virus antigen.

As used herein, “lentiviral vector” refers to a nucleic acid construct derived from a lentivirus, which is used to transduce a transgene containing a cis-acting lentiviral RNA or DNA sequence into a host cell. The lentiviral vector may be replication-defective, for example, it lacks the coding sequence of a functional lentiviral protein such as Gag, Pol, Rev, and/or Env protein. When the replication-deficient lentiviral vector is packaged into lentiviral particles, the lentiviral protein (for example, Gag, Pol, Rev and/or Env) needs to be provided in trans.

Lentiviral vectors can exist in the form of RNA or DNA. For example, the lentiviral vector may be in the form of a recombinant DNA molecule, such as a plasmid (also called a lentiviral transfer vector). A lentiviral vector can also refer to a genomic nucleic acid molecule contained in a complete lentiviral particle, which is a dimer of single-stranded RNA molecule. Lentiviral vector can also refer to a DNA sequence integrated into a host cell.

Lentiviral vectors can be derived from, for example, human immunodeficiency virus (HIV-1 or HIV-2), monkey immunodeficiency virus (SIV), equine infectious encephalitis virus (EIAV), goat arthritis encephalitis virus (CAEV), cattle Immunodeficiency virus (BIV) and Feline Immunodeficiency Virus (FIV), which have been modified to remove genetic determinants involved in pathogenicity and introduce foreign expression cassettes.

In some preferred embodiments, the lentiviral vector is a non-integrating lentiviral vector. Non-integrated lentiviral vectors can effectively avoid the potential risks of viral DNA integration into the human genome.

“Lentiviral particle” or “lentiviral vector particle” as used interchangeably herein refers to a packaged viral particle containing a lentiviral protein and its associated lentiviral genome (such as the lentiviral vector described herein), which it can infect host cells and express the protein encoded by the viral genome in the host.

The hepatitis B virus antigens according to the present invention may be antigens from hepatitis B virus of different genotypes or different serotypes. For example, the HBV can be selected from genotypes A, B, C, D, E, F, G, and H. Preferably, the HBV is genotype C. Alternatively, the HBV may be selected from serotypes ayw1, ayw2, ayw3, ayw4, ayr, adw2, adw4, adrq+ and adrq−. Preferably, the HBV is serotype adw or adr.

In some preferred embodiments, the HBV is genotype C, serotype adr. the hepatitis B virus antigen encoded in a lentiviral vector of the invention is selected from the group consisting of core antigen (HBcAg), PreS1 antigen (PreS1) and large S antigen (LargeS), or a combination thereof. In some preferred embodiments, the encoded hepatitis B virus antigen is a large S antigen (LargeS).

In some embodiments, the hepatitis B virus core antigen (HBcAg) comprises the amino acid sequence set forth as SEQ ID NO:1. In some embodiments, the coding nucleotide sequence of the hepatitis B virus core antigen (HBcAg) is codon-optimized for expression in humans. In some embodiments, the nucleotide sequence encoding the hepatitis B virus core antigen (HBcAg) is set forth as SEQ ID NO: 2 or 3.

In some embodiments, the encoded hepatitis B virus PreS1 antigen (PreS1) comprises the amino acid sequence set forth as SEQ ID NO: 4 or 7. In some embodiments, the coding nucleotide sequence of the hepatitis B virus PreS1 antigen (PreS1) is codon-optimized for expression in humans. In some embodiments, the coding nucleotide sequence of the hepatitis B virus PreS1 antigen (PreS1) is set forth as SEQ ID NO: 5, 6, 8 or 9, and in particular SEQ ID NO: 6 or 9.

In some embodiments, the encoded hepatitis B virus large S antigen (LargeS) comprises the amino acid sequence set forth as SEQ ID NO: 10 or 13. In some specific embodiments, the coding nucleotide sequence of the hepatitis B virus large S antigen (LargeS) is codon-optimized for expression in humans. In some embodiments, the coding nucleotide sequence of the hepatitis B virus large S antigen (LargeS) is set forth as SEQ ID NO: 11, 12, 14 or 15, and in particular SEQ ID NO: 12 or 15.

In some preferred embodiments, the encoded hepatitis B virus antigen is hepatitis B virus large S antigen (LargeS). In some preferred embodiments, the encoded hepatitis B virus large S antigen (LargeS) comprises the amino acid sequence set forth as SEQ ID NO:13. In some preferred embodiments, the coding nucleotide sequence of the hepatitis B virus large S antigen (LargeS) is set forth as SEQ ID NO:15.

In some embodiments, the nucleotide sequence encoding the hepatitis B virus antigen is operably linked to a regulatory element. In some embodiments, the regulatory element is a promoter. A suitable promoter may be the P2 microglobulin promoter (β2m), for example. Examples of P2m promoters can be found in International Patent Application Publication WO2013174630.

In some embodiments, the lentiviral vector further comprises one or more or all operably linked elements selected from the following: 5′LTR, y, RRE, cPPT/CTS, WPRE and 3′ LTR. In some embodiments, the 3′LTR lacks the U3 region (AU3).

In another aspect, the present invention provides a method of preparing a lentiviral vector particle containing a nucleotide sequence encoding a hepatitis B virus antigen, the method comprising:
a) co-transfecting the lentiviral vector of the present invention, one or more packaging vectors expressing Gag, Rev and/or Pol, and an envelope vector expressing an envelope protein into a suitable host cell, or transfecting the lentiviral vector of the present invention into suitable host cells capable of expressing Gag, Rev, Pol and/or envelope proteins;
b) culturing the transfected host cell to package the lentiviral vector into lentiviral vector particles; and
c) harvesting the lentiviral vector particles produced in step b).

In some embodiments, the suitable host cell capable of expressing Gag, Rev, Pol and/or envelope protein has been used to express Gag, Rev and/or Pol before transfecting the lentiviral vector of the present invention. Transfection of a variety of packaging vectors and envelope vectors expressing envelope proteins may also be performed.

Any other viral or non-viral envelope protein can be used for pseudotyped packaging, as long as the envelope protein is suitable for packaging and suitable for entering target cells. In some embodiments, the envelope protein is the envelope glycoprotein of vesicular stomatitis virus (VSV-G). For example, the envelope protein may be the vesicular stomatitis virus envelope protein of Indiana serotype (GenBank acc. No. J02428) or New Jersey serotype (GenBank acc. No. P04882).

It is known in this field and by those skilled in this field that one or more suitable packaging vectors expressing Gag, Rev and/or Pol and envelope vectors expressing envelope proteins suitable for packaging lentivirus can easily be obtained. In some embodiments, the aforementioned lentiviral vector is a plasmid.

Suitable host cells for preparing lentiviral vector particles include but are not limited to 293 cells, such as 293T cells.

The general method of constructing lentiviral vector and packaging of virus particles suitable for the present invention can be found in Chinese patents or patent applications CN101291688A, CN102083462B, CN104039968B, CN102083462B, etc.

In another aspect, the present invention provides lentiviral vector particles (also termed preparation of lentiviral particles) comprising a nucleotide sequence encoding a hepatitis B virus antigen, which comprises a lentiviral vector of the present invention or is prepared by the above-mentioned method of the present invention.

2. Disease Treatment and/or Prevention

In another aspect, the present invention provides that the lentiviral vector and/or lentiviral vector particles (also termed preparation of lentiviral particles) of the present invention are prepared for the treatment and/or prevention of hepatitis B virus infection or the treatment and/or prevention of diseases caused by hepatitis B virus infection in a subject in need thereof.

In another aspect, the present invention provides a pharmaceutical composition for treating and/or preventing hepatitis B virus infection or treating and/or preventing diseases caused by hepatitis B virus infection in a subject in need thereof, the composition comprising at least one lentiviral vector particle of the present invention, and a pharmaceutically acceptable carrier. In some embodiments, the pharmaceutical composition is a hepatitis B therapeutic vaccine.

As used herein, a “pharmaceutically acceptable carrier” is a substance that can be added to the active pharmaceutical ingredient to help formulate or stabilize the formulation without causing significant adverse toxicological effects to the patient, including but not limited to disintegrants, adhesives, fillers agents, buffers, isotonic agents, stabilizers, antioxidants, surfactants or lubricants.

“Disease caused by hepatitis B virus infection” includes but is not limited to hepatitis; liver cirrhosis; complications associated with liver cirrhosis, including ascites, esophageal varices, spontaneous peritonitis and hepatic encephalopathy; liver cancer; liver failure; diseases of the kidneys; inflammation or swelling of blood vessels; anemia; etc. caused by hepatitis B virus infection.

In some embodiments, the pharmaceutical composition includes two or more lentiviral vector particles of the present invention, each of which includes a different hepatitis B virus antigen-encoding nucleotide sequence. For example, the pharmaceutical composition may comprise two kinds of lentiviral vector particles of the present invention, wherein the first lentiviral vector particle comprises a nucleotide sequence encoding hepatitis B virus core antigen (HBcAg), and the second lentiviral vector particle comprises Hepatitis B virus PreS1 antigen (PreS1) encoding nucleotide sequence.

As used herein, the term “subject” refers to a mammal, such as a human. In some embodiments, the subject has been infected with a hepatitis B virus. In some embodiments, the subject is not infected with a hepatitis B virus. In some embodiments, the subject has been infected with a hepatitis B virus and has exhibited symptoms of disease caused by hepatitis B virus infection. In some embodiments, the subject has been infected with a hepatitis B virus, but does not exhibit symptoms of disease caused by hepatitis B virus infection.

In some embodiments, the hepatitis B virus may be selected from genotypes A, B, C, D, E, F, G, and H. Preferably, the hepatitis B virus is genotype C. In some embodiments, the hepatitis B virus may be selected from serotypes ayw1, ayw2, ayw3, ayw4, ayr, adw2, adw4, adrq+ and adrq−. Preferably, the hepatitis B virus is serotype adw or adr. In some preferred embodiments, the hepatitis B virus is genotype C, serotype adr.

“Therapeutically effective amount” or “therapeutically effective dose” refers to the amount of a substance, compound, material, or composition containing the compound that is at least sufficient to produce a therapeutic effect after administration to a subject. Therefore, it is the amount necessary to treat, prevent, cure, ameliorate, block or partially block the symptoms of the disease or disorder.

The “therapeutically effective amount” of the lentiviral vector particles of the present invention preferably results in a reduction in hepatitis B virus load, a reduction in the severity of hepatitis B symptoms, an increase in the frequency and duration of asymptomatic periods of the disease, or prevention of suffering caused by disease the injury or disability caused. For example, for the treatment of hepatitis B infection, a “therapeutically effective amount” preferably reduces the hepatitis B viral load by at least about 10%, preferably at least about 20%, and more preferably at least about 30%, relative to subjects not receiving treatment. More preferably at least about 40%, more preferably at least about 50%, more preferably at least about 60%, more preferably at least about 70%, more preferably at least about 80%, more preferably at least about 90%, more preferably at least about 95%, more preferably at least about 100%. Alternatively, the “therapeutically effective amount” preferably reduces the HBsAg level in the subject by at least about 10%, preferably at least about 20%, more preferably at least about 30%, more preferably at least about 40%, more preferably at least about 50%, more preferably at least about 60%, more preferably at least about 70%, more preferably at least about 80%, more preferably at least about 90%, more preferably at least about 95%, more preferably at least about 100%.

In some specific embodiments, the administration dose of the lentiviral vector particles of the present invention may include about 1×10⁷ TU/ml to about 1×10⁸ TU per dose, for example about 1×10⁷ TU, about 2×10⁷ TU, about 3×10⁷ TU, about 4×10⁷ TU, The lentiviral vector particles are about 5×10⁷ TU, about 6×10⁷ TU, about 7×10⁷ TU, about 8×10⁷ TU, about 9×10⁷ TU, about 10×10⁷ TU, preferably about 5×10⁷ TU. TU (transducing unit) refers to the number of virus particles with biological activity (for example, it can infect and enter target cells).

The lentiviral vector particles or pharmaceutical compositions of the present invention can be administered through one or more administration routes using one or more methods known in this field. Those skilled in this field understand that the route and/or manner of administration differ according to the desired result. Suitable routes of administration include but are not limited to intramuscular administration, subcutaneous administration, intradermal administration, oral administration, etc. In some embodiments, the lentiviral vector particles or pharmaceutical compositions of the present invention are administered by intramuscular injection and/or intravenous injection.

An exemplary treatment regimen of the lentiviral vector particles (also termed preparation of lentiviral vector particles) or pharmaceutical compositions of the present invention may be once a week, once every two weeks, once every three weeks, once every four weeks, once a month, once every 3 months, once every 3 months.-6 months once, or the initial dosing interval is slightly shorter (such as once a week to once every three weeks) and the later dosing interval is increased (such as once a month to once every 3-6 months). The lentiviral vector particles or pharmaceutical composition of the present invention can be administered once, twice, or more times. In some specific embodiments, the lentiviral vector particles or pharmaceutical composition of the present invention is/are administered twice, for example, once in the first week and once again in the second week.

### 3. LV Construction and Production Methods

For the construction of recombinant lentiviral vectors of the invention expressing core antigen (HBcAg), PreS1 antigen (PreS1), and/or Large S antigen (LargeS), sequences of HBcAg, PreS1 and large S derived from Hepatitis B virus strain FMC #97 were downloaded from GenBank and were codon optimized. The mammalian codon-optimized sequences coding for HBcAg, PreS1 and large S were cloned into the BamHI and XhoI restriction sites of the FLAP-SPlb2m-WPREm plasmid, to generate pFLAP-SP1b2m-transgene-WPREm. Plasmids were produced with maxiprep kits (Macherey-Nagel, Duren, Germany). Sequences were confirmed by double-stranded sequencing.

Lentiviral particles were produced by transient calcium phosphate co-transfection of HEK 293 T cells. Briefly, HEK 293 T cells were co-transfected with the plasmid vector pFLAP-SPlb2m-transgene-WPREm, a VSV-G envelope of Indiana serotype or New Jersey plasmid and an encapsidation pSD-GP-niNDK (D64V) plasmid. The viral particles were harvested 48 h post transfection and stored at −80 degrees. Viral titer was determined by transduction of HEK 293T cells with aphidicolin and followed by qPCR.

In another aspect, the present invention provides a virus production method for producing lentiviral vector particles, which comprises the lentiviral vector of the present invention, a suitable packaging vector, a suitable envelope vector and/or a suitable host cell such as 293 cell. The reagents may also include cell transfection reagents.

### Example 1. Construction of Candidate Hepatitis B Therapeutic Lentivirus Vaccine

### 1.1. Hepatitis B Genotype and Serotype Classification

Hepatitis B virus can undergo mutations in its genome nucleotide sequence during reproduction, and this mutation can sometimes lead to changes in the biological characteristics of the virus. Nucleotide differences between different genotypes of HBV can be used to track the route of HBV transmission, identify the source of infection, determine the transmission relationship and pathogenesis. At present, there are 10 HBV genotypes identified, and the geographical distribution of the 10 genotypes has been clear. Among them, genotype A is mainly distributed in northwestern Europe and Central Africa, genotypes B and C are common in Asia, genotype D is the dominant genotype in the Mediterranean region and the Middle East to India, and genotype E is mainly found in western Africa. Types F and H are more common in South America, and genotype G may originate in Central America. In most areas of China, patients infected with HBV are mainly B and C genotypes, accounting for more than 95%, while reports of A and D genotypes are only seen in some ethnic minority areas. The geographical distribution characteristics of HBV genotypes in China are also quite different. The C genotype is mainly prevalent in the north, with more than 90% in most areas, while the B genotype is prevalent in the south. Studies have shown that, compared with genotype B, genotype C infection has a higher risk of causing more serious liver diseases such as cirrhosis and liver cancer.

HBV can be divided into 9 serotypes: ayw1, ayw2, ayw3, ayw4, ayr, adw2, adw4, adrq+ and adrq−. The serotype only reflects the difference in amino acids of part of the envelope protein, and does not truly reflect the phylogenetic relationship of the virus, and a single nucleic acid change in the gene sequence may change the serotype, so the serotype does not truly reflect HBV differences in gene sequence. Studies have found that there is no strict correspondence between serotypes and genotypes. Different serotypes can belong to the same genotype, and the same serotype can be distributed in different genotypes (see Table 1). In China, it is mostly adw and adr subtypes.

TABLE 1
Correspondence between genotype, subgenotype and serotype

| Genotype | Serotype | Genome (bp) |
|---|---|---|
| A | adw2, adw1 | 3221 |
| B | adw2, ayw1 | 3215 |
| C | adr, ayr, adw2 | 3215 |
| D | ayw2, ayw3 | 3182 |
| E | ayw4 | 3212 |
| F | adw4, ayw4, adw2 | 3215 |
| G | adw2 | 3248 |
| H | adw3 | 3215 |
| I | adw | 3215 |
| J | ayw | 3182 |

### 1.2. Antigen Design and Codon Optimization

Three immunogens were selected as the research targets: core antigen (HBcAg), PreS1 antigen (PreS1 antigen) and Large S antigen (Large S antigen, including PreS1, PreS2 and surface antigen HBsAg). Since the main genotypes of HBV in China are type B and C, the genotype B, serotype adw HBV strain 536207 (GenBank: AY220698.1) and genotype C, serotype adr type HBV strain FMU #14 (GenBank: AF411408.1), its full-length sequence has a total of 3215 bases, of which positions 2848-3205 are preS1 coding genes, a total of 360 bases, and positions 2848-835 are Large S coding genes, a total of 1203 bases, Positions 1901-2452 are Core coding genes, with a total of 552 bases. According to the HBsAg amino acid sequence alignment of the genotype C HBV strain, it is found that the 204th amino acid is mostly Ser, and the 204th amino acid in this virus strain is Arg, so the adjusted codon AGA is AGC (highlighted in the sequence). In addition, due to the high homology of the Core gene, there are only four amino acid differences between different virus strains. After comparing the amino acid sequence of the core protein encoded by the B and C virus strains, the 5th, 83rd, 87th, and 97th amino acids were selected. The most frequently occurring amino acid coding sequence. The specific coding sequence of each antigen obtained is as follows:

HBV genotype B preS1 coding sequence
(SEQ ID NO: 5)
ATGGGAGGTTGGTCTTCCAAACCTCGAAAAGGCATGGGGACAAATCTTTC
TGTCCCCAATCCCCTGGGATTCTTCCCCGATCATCAGTTGGACCCTGCAT
TCAAAGCCAACTCAGAAAATCCAGATTGGGACCTCAACCCGCACAAGGAC
AACTGGCCGGACGCCAACAAGGTGGGAGTGGGAGCATTCGGGCCAGGGTT
CACCCCTCCCCATGGGGGACTGTTGGGGTGGAGCCCTCAGGCTCAGGGCC
TACTCACAACTGTGCCAGCAGCTCCTCCTCCTGCCTCCACCAATCGGCAG
TTAGGAAGGCAGCCTACTCCCTTATCTCCACCTCTAAGGGACACTCATCC
TCAGGCCTGA
HBV genotype B large S coding sequence
(SEQ ID NO: 11)
ATGGGAGGTTGGTCTTCCAAACCTCGAAAAGGCATGGGGACAAATCTTTC
TGTCCCCAATCCCCTGGGATTCTTCCCCGATCATCAGTTGGACCCTGCAT
TCAAAGCCAACTCAGAAAATCCAGATTGGGACCTCAACCCGCACAAGGAC
AACTGGCCGGACGCCAACAAGGTGGGAGTGGGAGCATTCGGGCCAGGGTT
CACCCCTCCCCATGGGGGACTGTTGGGGTGGAGCCCTCAGGCTCAGGGCC
TACTCACAACTGTGCCAGCAGCTCCTCCTCCTGCCTCCACCAATCGGCAG
TTAGGAAGGCAGCCTACTCCCTTATCTCCACCTCTAAGGGACACTCATCC
TCAGGCCATGCAGTGGAACTCCACCACTTTCCACCAAACTCTTCAAGATC
CCAGAGTCAGGGCCCTGTACTTTCCTGCTGGTGGCTCCAGTTCAGGAACA
GTGAGCCCTGCTCAAAATACTGTCTCTGCCATATCGTCAATCTTATCGAA
AACTGGGGACCCTGTACCGAACATGGAGAACATCGCATCAGGACTCCTAG
GACCCCTGCTCGTGTTACAGGCGGGGTTTTTCTTGTTGACAAAAATCCTC
ACAATACCACAGAGTCTAGACTCGTGGTGGACTTCTCTCAATTTTCTAGG
GGGAACACCCGTGTGTCTTGGCCAAAATTCGCAGTCCCAAATCTCCAGTC
ACTCACCAACCTGTTGTCCTCCAATTTGTCCTGGTTATCGCTGGATGTAT
CTGCGGCGTTTTATCATATTCCTCTGCATCCTGCTGCTATGCCTCATCTT
CTTGTTGGTTCTTCTGGACTATCAAGGTATGTTGCCCGTTTGTCCTCTAA
TTCCAGGATCATCAACAACCAGCACCGGACCATGCAAAACCTGCACGACT
CCTGCTCAAGGAACCTCTATGTTTCCCTCATGTTGCTGTACAAAACCTAC
GGACGGAAACTGCACCTGTATTCCCATCCCATCATCTTGGGCTTTCGCAA
AATTCCTATGGGAGTGGGCCTCAGTCCGTTTCTCTTGGCTCAGTTTACTA
GTGCCATTTGTTCAGTGGTTCGTAGGGCTTTCCCCCACTGTCTGGCTTTC
AGTTATATGGATGATTTGGTTTTGGGGGCCAAGTCTGTACAACATCTTGA
GTCCCTTTATGCCGCTGTTACCAATTTTCTTTTGTCTTTGGGTATACATT
TAA
HBV genotype B preS1 coding sequence
(SEQ ID NO: 8)
ATGGGAGGTTGGTCTTCCAAACCTCGAAAAGGCATGGGGACGAATCTTTC
TGTTCCCAATCCTCTGGGATTCTTTCCCGATCACCAGTTGGACCCTGCGT
TCGGAGCCAACTCAAACAATCCAGATTGGGACTTCAACCCCAACAAGGAT
CACTGGCCAGAGGCAAATCAGGTAGGAGCGGGAGCATTCGGGCCAGGGTT
CACCCCACCACACGGCGGTCTTTTGGGGTGGAGCCCTCAGGCTCAAGGCA
TATTGACAACAGTGCCAGTAGCACCTCCTCCTGCCTCCACCAATCGGCAG
TCAGGGAGACAGCCTACTCCCATCTCTCCACCTCTAAGAGACAGTCATCC
TCAGGCCTGA
HBV genotype C large S coding sequence
(SEQ ID NO: 14)
ATGGGAGGTTGGTCTTCCAAACCTCGAAAAGGCATGGGGACGAATCTTTC
TGTTCCCAATCCTCTGGGATTCTTTCCCGATCACCAGTTGGACCCTGCGT
TCGGAGCCAACTCAAACAATCCAGATTGGGACTTCAACCCCAACAAGGAT
CACTGGCCAGAGGCAAATCAGGTAGGAGCGGGAGCATTCGGGCCAGGGTT
CACCCCACCACACGGCGGTCTTTTGGGGTGGAGCCCTCAGGCTCAAGGCA
TATTGACAACAGTGCCAGTAGCACCTCCTCCTGCCTCCACCAATCGGCAG
TCAGGGAGACAGCCTACTCCCATCTCTCCACCTCTAAGAGACAGTCATCC
TCAGGCCATACAGTGGAATTCCACAACATTCCACCAAGCTCTGCTAGACC
CCAGAGTGAGGGGCCTATACTTTCCTGCTGGTGGCTCCAGTTCCGGAACA
GTAAACCCTGTTCCGACTACTGCCTCACCCACATCGTCAATCTTCTCGAG
GACTGGGGACCCTGCACCGAACATGGAGAACACAACATCAGGATTCCTAG
GACCCCTGCTCGTGTTACAGGCGGGGTTTTTCTTGTTGACAAGAATCCTC
ACAATACCACAGAGTCTAGACTCGTGGTGGACTTCTCTCAATTTTCTAGG
GGGAGCACCCACGTGTCCTGGCCAAAATTCGCAGTCCCCAACCTCCAATC
ACTCACCAACCTCTTGTCCTCCAATTTGTCCTGGCTATCGCTGGATGTGT
CTGCGGCGTTTTATCATATTCCTCTTCATCCTGCTGCTATGCCTCATCTT
CTTGTTGGTTCTTCTGGACTACCAAGGTATGTTGCCCGTTTGTCCTCTAC
TTCCAGGAACATCAACTACCAGCACAGGACCATGCAAGACCTGCACGATT
CCTGCTCAAGGAACCTCTATGTTTCCCTCTTGTTGCTGTACAAAACCTTC
GGACGGAAACTGCACTTGTATTCCCATCCCATCATCCTGGGCTTTCGCAA
GATTCCTATGGGAGTGGGCCTCAGTCCGTTTCTCCTGGCTCAGTTTACTA
GTGCCATTTGTTCAGTGGTTCGTAGGGCTTTCCCCCACTGTTTGGCTTTC
AGTTATATGGATGATGTGGTATTGGGGGCCAAGCCTGTACAACATCTTGA
GTCCCTTTTTACCTCTATTACCAATTTTCTTTTGTCTTTGGGTATACATT
TGA
HBV Core coding sequence
(SEQ ID NO: 2)
ATGGACATTGACCCGTATAAAGAATTTGGAGCTTCTGTGGAGTTACTCTC
TTTTTTGCCTTCTGACTTCTTTCCTTCTATTCGAGATCTCCTCGACACCG
CCTCTGCTCTGTATCGGGAGGCCTTAGAGTCTCCGGAACATTGTTCACCT
CACCATACGGCACTCAGGCAAGCTATTCTGTGTTGGGGTGAGTTAATGAA
TCTAGCCACCTGGGTGGGAAGTAATTTGGAAGATCCAGCATCCAGGGAAT
TAGTAGTCAGCTATGTCAACGTTAATATGGGCCTAAAAATCAGACAACTA
TTGTGGTTTCACATTTCCTGTCTTACTTTTGGGAGAGAAACTGTTCTTGA
ATATTTGGTGTCTTTTGGAGTGTGGATTCGCACTCCTCCCGCATATAGAC
CGCCAAATGCCCCTATCTTATCAACACTTCCGGAAACTACTGTTGTTAGA
CGAAGAGGCAGGTCCCCTAGAAGAAGAACTCCCTCGCCTCGCAGACGAAG
GTCTCAATCGCCGCGTCGCAGAAGATCTCAATCTCGGGAATCTCAATGTT
AG

The above coding sequence is provided to Suzhou GENEWIZ Company for gene synthesis. Before synthesis, according to the preference, GC content, high-level structure and exclusion of restriction sites in each host are optimized according to human codons. The optimized sequence:

HBV genotype B preS1 codon optimized sequence
(adw-preS1)
(SEQ ID NO: 6)
ATGGGCGGCTGGAGCAGCAAGCCCAGAAAGGGCATGGGCACCAACCTGAG
CGTGCCCAACCCCCTGGGCTTCTTCCCCGACCACCAGCTGGACCCCGCCT
TCAAGGCCAACAGCGAGAACCCCGACTGGGACCTGAACCCCCACAAGGAC
AACTGGCCTGACGCCAACAAGGTGGGCGTGGGAGCTTTCGGCCCTGGCTT
CACCCCTCCCCATGGAGGACTGCTGGGCTGGAGCCCTCAGGCTCAGGGAC
TGCTGACCACAGTGCCCGCTGCTCCTCCTCCTGCCAGCACCAACAGGCAG
CTGGGCAGACAGCCCACACCCCTGAGCCCTCCTCTGAGAGACACCCACCC
CCAGGCCTGA
HBV genotype B Large S codon optimized sequence
(adw-LargeS)
(SEQ ID NO: 12)
ATGGGAGGCTGGTCCTCCAAACCCAGGAAGGGCATGGGCACAAACCTGTC
CGTGCCCAACCCTCTGGGCTTTTTCCCCGACCACCAGCTGGACCCCGCCT
TCAAGGCTAACAGCGAGAACCCCGACTGGGACCTGAATCCCCACAAGGAC
AATTGGCCCGATGCCAATAAGGTGGGCGTGGGCGCCTTCGGCCCTGGATT
TACACCCCCCCATGGAGGACTGCTGGGATGGTCCCCTCAGGCCCAAGGCC
TGCTGACCACAGTGCCCGCTGCTCCTCCCCCTGCTTCCACCAATAGACAG
CTCGGCAGACAGCCCACACCCCTGTCCCCTCCTCTGAGGGACACCCATCC
CCAGGCCATGCAGTGGAATAGCACCACCTTCCACCAGACACTGCAGGACC
CCAGGGTGAGAGCCCTGTACTTCCCCGCCGGAGGTTCTAGCAGCGGAACA
GTGAGCCCCGCCCAGAACACAGTGTCCGCCATCAGCAGCATTCTGTCCAA
GACAGGCGACCCCGTGCCCAACATGGAGAACATCGCCAGCGGACTCCTGG
GACCTCTCCTGGTGCTGCAGGCCGGCTTCTTCCTGCTGACCAAGATCCTG
ACCATCCCCCAGAGCCTGGATTCCTGGTGGACCAGCCTGAACTTTCTGGG
AGGCACCCCCGTGTGCCTGGGCCAGAATAGCCAGAGCCAGATCTCCTCCC
ACAGCCCTACCTGCTGCCCCCCTATCTGCCCTGGATACAGGTGGATGTAC
CTGAGGAGGTTCATCATCTTCCTGTGCATTCTGCTGCTGTGCCTCATCTT
TCTGCTGGTGCTGCTGGATTACCAGGGCATGCTGCCTGTGTGTCCCCTGA
TCCCCGGCAGCAGCACCACAAGCACCGGCCCCTGTAAGACCTGTACCACC
CCCGCCCAGGGAACCTCCATGTTCCCTTCCTGCTGCTGCACCAAGCCCAC
CGACGGCAACTGTACATGCATCCCCATTCCCAGCAGCTGGGCCTTTGCTA
AATTCCTGTGGGAGTGGGCCTCCGTGAGATTCAGCTGGCTGTCCCTGCTG
GTGCCTTTCGTGCAGTGGTTCGTGGGACTGTCCCCCACAGTGTGGCTGTC
CGTGATCTGGATGATCTGGTTCTGGGGCCCCAGCCTGTACAACATCCTGA
GCCCTTTCATGCCCCTGCTGCCCATCTTCTTTTGCCTCTGGGTGTACATT
TGA
HBV genotype C preS1 codon optimized sequence
(adr-preS1)
(SEQ ID NO: 9)
ATGGGCGGATGGTCCTCCAAGCCTAGAAAGGGCATGGGCACCAATCTGTC
CGTGCCCAACCCCCTGGGCTTTTTCCCCGATCACCAGTTAGATCCTGCCT
TCGGCGCCAACAGCAACAACCCTGACTGGGACTTCAACCCCAACAAGGAT
CACTGGCCCGAGGCCAATCAAGTGGGCGCTGGAGCTTTCGGCCCTGGCTT
CACACCTCCCCATGGAGGACTGCTGGGCTGGTCCCCTCAGGCCCAGGGAA
TTCTCACAACAGTGCCCGTGGCCCCTCCTCCCGCTAGCACCAACAGGCAG
AGCGGCAGGCAGCCCACACCTATCAGCCCCCCTCTGAGAGATTCCCACCC
CCAGGCCTGA
HBV genotype C Large S codon optimized sequence
(adr-LargeS)
(SEQ ID NO: 15)
ATGGGCGGATGGTCCTCCAAGCCTAGAAAGGGCATGGGCACCAATCTGTC
CGTGCCCAACCCCCTGGGCTTTTTCCCCGATCACCAGTTAGATCCTGCCT
TCGGCGCCAACAGCAACAACCCTGACTGGGACTTCAACCCCAACAAGGAT
CACTGGCCCGAGGCCAATCAAGTGGGCGCTGGAGCTTTCGGCCCTGGCTT
CACACCTCCCCATGGAGGACTGCTGGGCTGGTCCCCTCAGGCCCAGGGAA
TTCTCACAACAGTGCCCGTGGCCCCTCCTCCCGCTAGCACCAACAGGCAG
AGCGGCAGGCAGCCCACACCTATCAGCCCCCCTCTGAGAGATTCCCACCC
CCAGGCCATCCAGTGGAATAGCACCACCTTCCACCAAGCCCTGCTCGACC
CTAGGGTGAGGGGCCTGTACTTTCCCGCTGGCGGCAGCTCCAGCGGCACA
GTGAATCCCGTGCCCACAACCGCCTCCCCTACCTCCTCCATTTTCAGCAG
AACCGGCGACCCCGCCCCCAACATGGAGAACACAACCTCCGGCTTTCTCG
GCCCTCTGCTGGTGCTGCAGGCCGGCTTCTTCCTGCTGACCCGTATTTTA
ACCATCCCCCAGAGCCTGGACTCCTGGTGGACCTCCCTGAACTTCCTGGG
AGGAGCCCCTACCTGTCCCGGACAGAACTCCCAGTCCCCTACCAGCAATC
ACTCCCCCACCAGCTGCCCTCCTATCTGCCCCGGCTACAGGTGGATGTGT
CTGAGGAGGTTCATCATCTTCCTGTTCATCCTGCTGCTGTGCCTCATTTT
CCTGCTGGTGCTGCTGGACTACCAGGGCATGCTGCCCGTGTGTCCTCTGC
TGCCCGGCACAAGCACCACAAGCACCGGCCCCTGCAAGACCTGTACCATC
CCCGCCCAGGGCACCTCCATGTTTCCCTCCTGCTGCTGCACCAAGCCCTC
CGATGGCAACTGCACCTGCATCCCTATCCCCTCCTCCTGGGCCTTCGCCA
GATTCCTGTGGGAATGGGCTTCCGTGAGGTTCTCCTGGCTGTCCCTGCTG
GTGCCCTTTGTGCAGTGGTTTGTGGGCCTGAGCCCTACAGTGTGGCTGTC
CGTGATCTGGATGATGTGGTATTGGGGCCCCTCCCTGTACAACATCCTGA
GCCCCTTCCTGCCCCTGCTGCCCATCTTCTTCTGCCTGTGGGTGTACATC
TGA
HBV Core codon optimized sequence
(SEQ ID NO: 3)
ATGGACATCGACCCTTACAAGGAGTTCGGCGCCAGCGTGGAGCTCCTGAG
CTTCCTGCCCAGCGACTTCTTTCCCAGCATCAGAGACCTGCTGGACACCG
CCAGCGCCCTGTACAGGGAAGCCCTGGAGAGCCCCGAGCACTGTAGCCCT
CACCACACCGCCCTCAGACAGGCCATCCTGTGCTGGGGCGAGCTGATGAA
CCTGGCCACCTGGGTGGGAAGCAACCTGGAAGACCCCGCCTCCAGGGAGC
TGGTGGTGTCCTACGTGAACGTGAACATGGGCCTGAAGATCAGGCAGCTG
CTGTGGTTCCACATCAGCTGCCTGACCTTCGGCAGAGAAACCGTGCTGGA
GTACCTGGTGAGCTTCGGCGTCTGGATCAGAACCCCTCCTGCCTACAGAC
CCCCTAACGCCCCCATCCTGTCCACCCTGCCCGAGACCACAGTGGTGAGG
AGGAGAGGCAGGAGCCCCAGAAGGAGGACCCCTAGCCCCAGGAGGAGGAG
AAGCCAGTCCCCCAGGAGAAGAAGGTCCCAGTCCAGGGAGAGCCAGTGCT
GA

The 5′ of each sequence was inserted into the BamH I restriction site GGATCC, and the 3′ was inserted into the XhoI restriction site CTCGAG. After synthesis, the target gene sequence was constructed into the eukaryotic expression vector pcDNA3.1(+). Sequencing verified that the immunogen gene was correct in full length. The amino acid sequence of the selected antigen is as follows:

HBV Core amino acid sequence
(SEQ ID NO: 1)
MDIDPYKEFGASVELLSFLPSDFFPSIRDLLDTASALYREALESPEHCSP
HHTALRQAILCWGELMNLATWVGSNLEDPASRELVVSYVNVNMGLKIRQL
LWFHISCLTFGRETVLEYLVSFGVWIRTPPAYRPPNAPILSTLPETTVVR
RRGRSPRRRTPSPRRRRSQSPRRRRSQSRESQC
HBV type B preS1 amino acid sequence
(SEQ ID NO: 4)
MGGWSSKPRKGMGTNLSVPNPLGFFPDHQLDPAFKANSENPDWDLNPHKD
NWPDANKVGVGAFGPGFTPPHGGLLGWSPQAQGLLTTVPAAPPPASTNRQ
LGRQPTPLSPPLRDTHPQA
HBV Type C preS1 amino acid sequence
(SEQ ID NO: 7)
MGGWSSKPRKGMGTNLSVPNPLGFFPDHQLDPAFGANSNNPDWDFNPNKD
HWPEANQVGAGAFGPGFTPPHGGLLGWSPQAQGILTTVPVAPPPASTNRQ
SGRQPTPISPPLRDSHPQA
HBV Type B Large S amino acid sequence
(SEQ ID NO: 10)
MGGWSSKPRKGMGTNLSVPNPLGFFPDHQLDPAFKANSENPDWDLNPHKD
NWPDANKVGVGAFGPGFTPPHGGLLGWSPQAQGLLTTVPAAPPPASTNRQ
LGRQPTPLSPPLRDTHPQAMQWNSTTFHQTLQDPRVRALYFPAGGSSSGT
VSPAQNTVSAISSILSKTGDPVPNMENIASGLLGPLLVLQAGFFLLTKIL
TIPQSLDSWWTSLNFLGGTPVCLGQNSQSQISSHSPTCCPPICPGYRWMY
LRRFIIFLCILLLCLIFLLVLLDYQGMLPVCPLIPGSSTTSTGPCKTCTT
PAQGTSMFPSCCCTKPTDGNCTCIPIPSSWAFAKFLWEWASVRFSWLSLL
VPFVQWFVGLSPTVWLSVIWMIWFWGPSLYNILSPFMPLLPIFFCLWVYI
HBV Type C Large S amino acid sequence
(SEQ ID NO: 13)
MGGWSSKPRKGMGTNLSVPNPLGFFPDHQLDPAFGANSNNPDWDFNPNKD
HWPEANQVGAGAFGPGFTPPHGGLLGWSPQAQGILTTVPVAPPPASTNRQ
SGRQPTPISPPLRDSHPQAIQWNSTTFHQALLDPRVRGLYFPAGGSSSGT
VNPVPTTASPTSSIFSRTGDPAPNMENTTSGFLGPLLVLQAGFFLLTRIL
TIPQSLDSWWTSLNFLGGAPTCPGQNSQSPTSNHSPTSCPPICPGYRWMC
LRRFIIFLFILLLCLIFLLVLLDYQGMLPVCPLLPGTSTTSTGPCKTCTI
PAQGTSMFPSCCCTKPSDGNCTCIPIPSSWAFARFLWEWASVRFSWLSLL
VPFVQWFVGLSPTVWLSVIWMMWYWGPSLYNILSPFLPLLPIFFCLWVYI

### 1.3. Lentivirus Construction

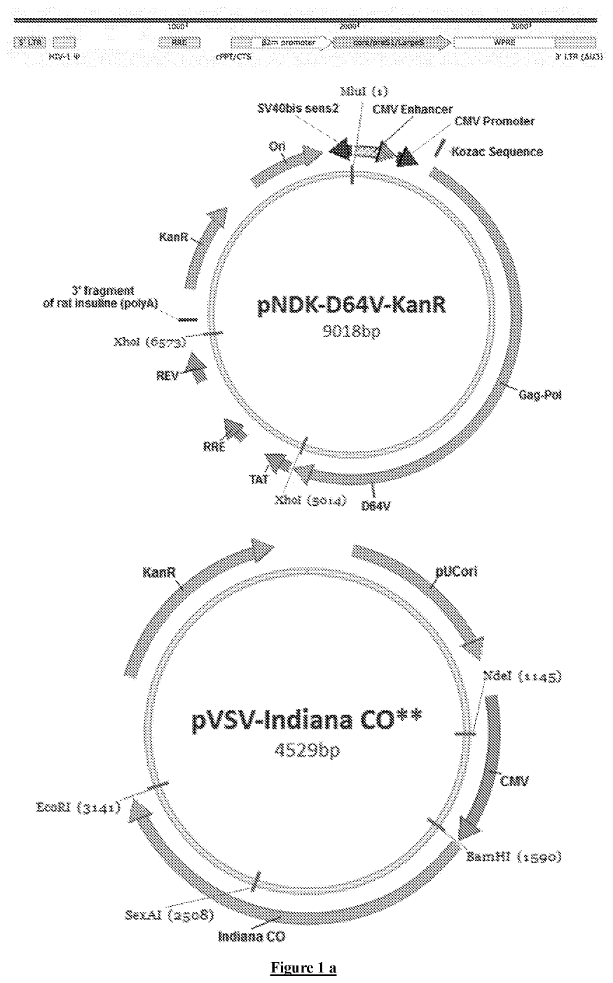

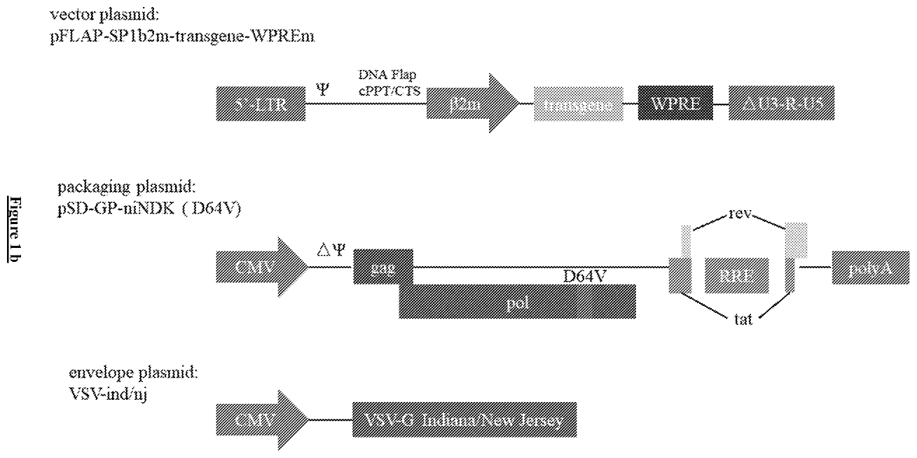

The recombinant plasmids containing HBV type C preS1, large S and core antigens were co-transfected with packaging vector and envelope vector to carry out lentivirus packaging. The plasmid vector structure used is shown in FIG. 1. The corresponding serial numbers of the synthesized lentivirus are JW27 (comprising the HBV Core codon optimized nucleic acid sequence set forth as SEQ ID NO: 3 expressing the amino acid sequence set forth as SEQ ID NO: 1), JW28 (comprising HBV genotype C preS1 codon optimized nucleic acid sequence set forth as SEQ ID NO: 9 expressing the amino acid sequence set forth as SEQ ID NO: 7) and JW29 (comprising HBV genotype C Large S codon optimized nucleic acid sequence set forth as SEQ ID NO: 15 expressing the amino acid sequence set forth as SEQ ID NO: 13). In addition, the control group is a 10 lentivirus with green fluorescent protein GFP inserted, named JW30, and the inserted GFP sequence is as follows:

GFP coding sequence
(SEQ ID NO: 16)
ATGAGTAAAGGAGAAGAACTTTTCACTGGAGTTGTCCCAATTCTTGTTGA
ATTAGATGGTGATGTTAATGGGCACAAATTTTCTGTCAGTGGAGAGGGTG
AAGGTGATGCAACATACGGAAAACTTACCCTTAAATTTATTTGCACTACT
GGAAAACTACCTGTTCCATGGCCAACACTTGTCACTACTTTCGGTTATGG
TGTTCAATGCTTTGCGAGATACCCAGATCATATGAAACAGCATGACTTTT
TCAAGAGTGCCATGCCTGAAGGTTATGTACAGGAAAGAACTATATTTTTC
AAAGATGACGGGAACTACAAGACACGTGCTGAAGTCAAGTTTGAAGGTGA
TACCCTTGTTAATAGAATCGAGTTAAAAGGTATTGATTTTAAAGAAGATG
GAAACATTCTTGGACACAAATTGGAATACAACTATAACTCACACAATGTA
TACATCATGGCAGACAAACAAAAGAATGGAATCAAAGTTAACTTCAAAAT
TAGACACAACATTGAAGATGGAAGCGTTCAACTAGCAGACCATTATCAAC
AAAATACTCCAATTGGCGATGGCCCTGTCCTTTTACCAGACAACCATTAC
CTGTCCACACAATCTGCCCTTTCGAAAGATCCCAACGAAAAGAGAGACCA
CATGGTCCTTCTTGAGTTTGTAACAGCTGCTGGGATTACACATGGCATGG
ATGAACTATACAAATAA

### Example 2. Evaluation of the Effect of Hepatitis B Therapeutic Lentivirus Vaccine in AAV8-1.3HBV Mouse Model

### 2.1 Experimental Method

### Establishment of Mice HBV Infection Model

C57bl/6j mice aged 6-8 weeks or weighing about 20 g were used. The mice were purchased from Beijing Weitonglihua Company. After the purchase, they were adapted to the environment in the animal room for about 1 week. AAV8-1.3HBV 5.10¹⁰ GC (genome copy)/mouse was injected into the tail vein, the injection volume is 200 μL. The usedAAV8-1.3HBV (purchased from Guangzhou Paizhen Biotechnology Co., Ltd.) is a genotype D HBV, and its genome sequence is the sequence shown in SEQ ID NO:17.

### Hepatitis B Therapeutic Lentivirus Vaccine Immunization

Mice were injected with AAV8-1.3HBV into the tail vein for 4 weeks to establish a persistent infection model. The mice were injected with JW27, JW28, JW29 and JW30 lentiviruses into the hind leg muscles. The injection volume was 5.10⁷ TU and the injection volume was 50 μL. JW27 is a lentivirus expressing HBV genotype C core antigen, JW28 is a lentivirus expressing HBV genotype C preS1 antigen, JW29 is a lentivirus expressing HBV genotype C Large S antigen (preS1+preS2+HBsAg) antigen, JW30 is Control lentivirus expressing GFP.

### HBsAg Detection

Add 3 μL serum to 297 μL PBS to prepare a 100× dilution, and send the dilution to Jinyu Medical for HBsAg detection. The detection kit is the Abbott Hepatitis B Virus Surface Antigen Quantitative Determination Kit (Chemiluminescence Microparticle Immunoassay Method), the detection limit is 5 IU/mL.

### Mice Serum HBV-DNA Detection

Use Hunan Shengxiang Biomedical's Viral Nucleic Acid Quantitative Determination Kit (one-step method), and the operation steps are performed in accordance with the instructions.

### Anti-HBs Detection

Add 3 μl serum to 297 μL PBS to prepare a 100× dilution, and send the dilution to Jinyu Medical for anti-HBs detection. The detection kit is Abbott Hepatitis B Virus Surface Antibody Quantitative Determination Kit (Chemiluminescence Microparticles Immunoassay), the detection limit is 10 IU/L.

### HE and IHC Detection of Liver Tissue

The mice were anesthetized by intraperitoneal injection of chloral hydrate. After the mice were deeply anesthetized, the chest cavity of the mice was opened with scissors, and 5 ml PBS was injected from the apex of the heart to observe the color change of the liver until the liver turned white. Take a part of the liver tissue put it in formalin fixative, transfer to Ribiology (Shanghai Ruibaohe Biotechnology Co., Ltd.) for paraffin embedding, and then perform HE staining and HBcAg+ staining.

### Mice IFN-Gamma Elispot Detection

### Single Cell Acquisition and Stimulation:

After dislocation of the cervical spine of the mouse, soak it in 75% alcohol for 5 minutes; open the abdominal cavity of the mouse, remove the spleen, and put it in 5 mL 1640 medium; grind the spleen with gauze, and transfer the ground liquid to a 15 mL centrifuge tube Centrifuge at 600 g for 5 min, discard the supernatant; resuspend with 3 ml of lysate and mix well; after lysis for 3 min, add 5 mL of 1640 medium containing 10% FCS; centrifuge at 600 g for 5 min, discard the supernatant; resuspend with 1 ml of complete medium Count after suspension; use peptide library for stimulation, respectively CORE, PreS1, PreS2 and HBsAg, the final working concentration of each peptide is 2 ug/mL, use PMA+Ionomycin stimulation as a positive control.

### Cell Function IFN-Gamma Elispot Test:

(1) Add cells to a 96-well plate at a concentration of 1×10⁶/100 μL, 100 μL per well, add 100 μL of IL-2 stimulating peptide as required, and the final concentration of each peptide is 2 μg/mL, incubate for 6 days at 37° C., 5% carbon dioxide, and change half of the medium on the third day, and add the same concentration of IL-2 and stimulating peptide.

(2) Elispot plate antibody overnight coating: Dilute the anti-IFN-gamma antibody 1:200 (as recommended by the instructions) in sterile PBS according to the number of wells required for the experiment, add 100 μL/well to the Elispot plate, and incubate overnight at 4° C.

(3) Take out the Elispot plate that has been incubated overnight, discard the liquid in the well, and add 200 μL of complete medium (wash once, let it stand for 3 minutes, and discard. Add 200 μL of complete medium and seal at room temperature for 2 hours.

(4) After closing, discard the culture medium, and suspend the cells of the control group and the treatment group to a final volume of 100-150 μL, plus positive and negative well controls. After mixing gently, place in a humid box and incubate in a 37° C., 5% carbon dioxide incubator for about 24 hours.

(5) Discard the culture. Add 200 μL of distilled water/well to wash twice, leave it for 3-5 minutes each time, and then discard it. Pat dry the remaining liquid on absorbent paper. Add 200 L/well PBS+0.05% Tween and wash 3 times.

(6) Dilute the biotin-conjugated detection antibody 1:250 in PBS+10% NCS, add 100 μL/well, and incubate at room temperature for 2 hours.

(7) Discard the liquid, add 200 μL/well PBS+0.05% Tween and wash 3 times. Discard after 1-2 min each time.

(8) Dilute Streptavidin-HRP in PBS+10% NCS at a ratio of 1:100, add 100 μL/well, and incubate at room temperature for 1 hour.

(9) Discard the reaction solution, add 200 μL/well PBS+0.05% Tween and wash 4 times. Discard after 1-2 min each time. Add 200 μL/well PBS, wash twice, and discard.

(10) Add 1 drop of chromogenic substrate per ml of chromogenic buffer. After mixing, add it to 100 μL wells. React at room temperature and avoid light for 5-60 min. When a clearer red spot appears, gently rinse the board in tap water for 5 minutes to stop the reaction.

(11) Let the board dry naturally.

### Experimental Design

The experimental timeline is shown in FIG. 2. The experimental schedule is shown in Table 2. The third injection of JW27, JW28 and JW30 in the tenth week was immunized with the lentivirus coated with New Jersey VSV-G. Serum was collected weekly for HBsAg, HBeAg and HBV-DNA detection.

TABLE 2
Experiment schedule

| Week | Test Content |
|---|---|
| 0 | Mouse tail vein injection of AAV8-1.3HBV |
| 4 | Collect peripheral blood and separate serum to |
| | identify the modeling situation |
| 4 | First injection of LV intramuscularly |
| 5 | Collect peripheral blood and perform HBV |
| | related index detection; intramuscular |
| | injection of the second LV |
| 6 | Collect peripheral blood and detect HBV |
| | related indicators |
| 7 | Collect peripheral blood and perform HBV |
| | related index detection; |
| | One male mouse (No. 192) died in JW27 group |
| 8 | Collect peripheral blood and detect HBV |
| | related indicators |
| 9 | Collect peripheral blood and detect HBV |
| | related indicators |
| 10 | Collect peripheral blood and perform HBV |
| | related index detection; |
| | The four groups of mice of JW-27, JW-28, |
| | JW-27 + JW-28 and JW-30 were boosted with |
| | different envelope vaccines |
| 11 | Collect peripheral blood and detect HBV |
| | related indicators |
| 12 | Collect peripheral blood and detect HBV |
| | related indicators |
| 13 | Collect peripheral blood and detect HBV |
| | related indicators |
| 15 | Collect peripheral blood and detect HBV |
| | related indicators |
| 19 | Collect peripheral blood and detect HBV |
| | related indicators |
| 23 | Collect peripheral blood and detect HBV |
| | related indicators |
| 27 | Collect peripheral blood and detect HBV |
| | related indicators |
| 34 | Collect peripheral blood and detect HBV |
| | related indicators |
| 38 | Collect peripheral blood and detect HBV |
| | related indicators |
| 42 | Take splenocytes and bone marrow cells of |
| | two mice in group JW29 and adopt transfer 5 |
| | AAV8-1.3HBV mice in JW30 group |
| 44 | The mice were sacrificed, liver and spleen |
| | were taken, IFN-γ ELISpot detection, |
| | immunohistochemistry and staining were |
| | performed |

### 2.2 Experimental Results

### HBV Mice Model

In this experiment, 5E+10 GC (genome copies) AAV8-1.3HBV virus was injected through the tail vein into 100 mice, 50 male and female, numbered 174-273 (174-223 for males, 224-273 for females). Two weeks later, 10 males and 10 males were randomly selected for HBsAg and HBV DNA testing to verify whether the mice were infected with HBV. The results are shown in Table 3 and Table 4 below.

TABLE 3
Mice HBsAg detection

| | | Number | |
|---|---|---|---|
| Number (male) | HBsAg (IU/mL) | (Female) | HBsAg (IU/mL) |
| 184 | 809 | 234 | 691 |
| 188 | 1666 | 236 | 757 |
| 189 | 1967 | 237 | 1758 |
| 196 | 1597 | 240 | 300 |
| 201 | 2134 | 243 | 884 |
| 208 | 2169 | 249 | 679 |
| 214 | 1357 | 254 | 498 |
| 216 | 2094 | 255 | 905 |
| 217 | 1235 | 264 | 366 |
| 220 | 1781 | 269 | 80 |

TABLE 4
Mice HBV DNA detection

| | HBV DNA | Number | HBV DNA |
|---|---|---|---|
| Number (male) | (Log₁₀ IU/mL) | (Female) | (Log₁₀ IU/mL) |
| 184 | 3.0869 | 234 | 2.5744 |
| 188 | 3.7102 | 236 | 2.1173 |
| 189 | 4.5343 | 237 | 1.5667 |
| 196 | 3.6374 | 240 | 1.9268 |
| 201 | 4.1395 | 243 | 2.8272 |
| 208 | 4.4408 | 249 | 3.0592 |
| 214 | 3.5543 | 254 | 2.7995 |
| 216 | 3.5716 | 255 | 3.2427 |
| 217 | 4.3819 | 264 | 2.7302 |
| 220 | 4.1188 | 269 | 3.2563 |

The results show that AAV8-1.3HBV has successfully caused infection in the selected mice. At 4 weeks, 100 mice were tested for HBsAg and HBV DNA. According to the data of HBsAg (Table 5) and HBV DNA (Table 6), it can be seen that all 100 mice have been successfully infected with HBV.

TABLE 5
Mice HBsAg detection

| | | Number | |
|---|---|---|---|
| Number (male) | HBsAg (IU/mL) | (Female) | HBsAg (IU/mL) |
| 174 | 1846 | 224 | 297 |
| 175 | 196 | 225 | 377 |
| 176 | 29 | 226 | 418 |
| 177 | 869 | 227 | 438 |
| 178 | 236 | 228 | 480 |
| 179 | 1612 | 229 | 217 |
| 180 | 172 | 230 | 15 |
| 181 | 745 | 231 | 190 |
| 182 | 1063 | 232 | 417 |
| 183 | 1625 | 233 | 514 |
| 184 | 152 | 234 | 485 |
| 185 | 662 | 235 | 135 |
| 186 | 148 | 236 | 345 |
| 187 | 1253 | 237 | 443 |
| 188 | 1542 | 238 | 278 |
| 189 | 1586 | 239 | 603 |
| 190 | 1055 | 240 | 140 |
| 191 | 1388 | 241 | 363 |
| 192 | 832 | 242 | 345 |
| 193 | 619 | 243 | 331 |
| 194 | 103 | 244 | 119 |
| 195 | 601 | 245 | 204 |
| 196 | 1644 | 246 | 418 |
| 197 | 652 | 247 | 699 |
| 198 | 585 | 248 | 65 |
| 199 | 1066 | 249 | 449 |
| 200 | 1023 | 250 | 269 |
| 201 | 1325 | 251 | 246 |
| 202 | 451 | 252 | 149 |
| 203 | 935 | 253 | 297 |
| 204 | 86 | 254 | 306 |
| 205 | 587 | 255 | 629 |
| 206 | 367 | 256 | 484 |
| 207 | 193 | 257 | 201 |
| 208 | 396 | 258 | 389 |
| 209 | 572 | 259 | 315 |
| 210 | 743 | 260 | 345 |
| 211 | 903 | 261 | 233 |
| 212 | 206 | 262 | 285 |
| 213 | 501 | 263 | 477 |
| 214 | 707 | 264 | 405 |
| 215 | 894 | 265 | 463 |
| 216 | 1370 | 266 | 640 |
| 217 | 893 | 267 | 341 |
| 218 | 1278 | 268 | 244 |
| 219 | 907 | 269 | 413 |
| 220 | 997 | 270 | 299 |
| 221 | 1149 | 271 | 229 |
| 222 | 344 | 272 | 171 |
| 223 | 1613 | 273 | 351 |

TABLE 6
Mice HBV DNA detection

| | HBV DNA | Number | HBV DNA |
|---|---|---|---|
| Number (male) | (Log₁₀ IU/mL) | (Female) | (Log₁₀ IU/mL) |
| 174 | 4.89 | 224 | 3.53 |
| 175 | 2.80 | 225 | 3.30 |
| 176 | 2.15 | 226 | 3.10 |
| 177 | 2.96 | 227 | 3.36 |
| 178 | 3.21 | 228 | 2.07 |
| 179 | 4.81 | 229 | 3.25 |
| 180 | 2.69 | 230 | 2.04 |
| 181 | 3.68 | 231 | 2.95 |
| 182 | 3.50 | 232 | 3.39 |
| 183 | 4.10 | 233 | 3.48 |
| 184 | 2.41 | 234 | 3.77 |
| 185 | 2.81 | 235 | 2.80 |
| 186 | 2.65 | 236 | 3.09 |
| 187 | 3.56 | 237 | 3.11 |
| 188 | 3.37 | 238 | 2.82 |
| 189 | 5.38 | 239 | 3.91 |
| 190 | 4.75 | 240 | 3.00 |
| 191 | 5.10 | 241 | 3.34 |
| 192 | 3.92 | 242 | 2.46 |
| 193 | 4.28 | 243 | 3.97 |
| 194 | 3.68 | 244 | 2.26 |
| 195 | 2.44 | 245 | 2.95 |
| 196 | 3.19 | 246 | 3.37 |
| 197 | 3.74 | 247 | 4.10 |
| 198 | 3.78 | 248 | 3.25 |
| 199 | 4.36 | 249 | 2.93 |
| 200 | 3.93 | 250 | 2.19 |
| 201 | 4.45 | 251 | 2.27 |
| 202 | 2.65 | 252 | 2.12 |
| 203 | 4.28 | 253 | 1.95 |
| 204 | 3.32 | 254 | 2.02 |
| 205 | 3.91 | 255 | 2.25 |
| 206 | 4.33 | 256 | 2.34 |
| 207 | 3.32 | 257 | 0.78 |
| 208 | 4.48 | 258 | 2.51 |
| 209 | 3.44 | 259 | 2.76 |
| 210 | 3.82 | 260 | 3.50 |
| 211 | 4.06 | 261 | 2.55 |
| 212 | 3.46 | 262 | 2.37 |
| 213 | 2.90 | 263 | 2.98 |
| 214 | 2.74 | 264 | 3.49 |
| 215 | 2.90 | 265 | 2.86 |
| 216 | 3.17 | 266 | 2.89 |
| 217 | 3.77 | 267 | 2.45 |
| 218 | 3.05 | 268 | 3.05 |
| 219 | 3.15 | 269 | 3.48 |
| 220 | 3.78 | 270 | 2.82 |
| 221 | 3.36 | 271 | 2.28 |
| 222 | 3.13 | 272 | 2.91 |
| 223 | 4.33 | 273 | 3.16 |

### Grouping

Due to individual differences, the levels of HBsAg and HBV DNA in mice are not uniform. In order to reduce differences between individuals, 25 mice with similar surface antigens and viral parameters were selected from male and female mice, and randomly divided into 5 groups, each with 5 male and 5 female mice are grouped as shown in Table 7:

TABLE 7
Experimental grouping

| Group | Female | Male | Group | Female | Male |
|---|---|---|---|---|---|
| JW27 | 233 | 179 | JW28 | 266 | 196 |
| | 237 | 216 | | 263 | 188 |
| | 269 | 182 | | 264 | 199 |
| | 273 | 220 | | 225 | 190 |
| | 259 | 192 | | 224 | 217 |
| JW27 + 28 | 234 | 174 | JW29 | 247 | 223 |
| | 265 | 201 | | 249 | 191 |
| | 246 | 187 | | 232 | 218 |
| | 236 | 219 | | 241 | 203 |
| | 270 | 210 | | 268 | 211 |
| JW30 | 239 | 183 | | | |
| | 227 | 189 | | | |
| | 226 | 221 | | | |
| | 260 | 200 | | | |
| | 243 | 181 | | | |

Immunization with Therapeutic Lentiviral Vaccine

After confirming the establishment of the persistent HBV infection model in mice, the 5 groups were immunized with therapeutic vaccine, and the 5 groups were injected intramuscularly with LV-JW27, LV-JW28, LV-JW27+28, LV-JW29 and LV-JW30. The vaccine injection dose is 5E+7 TU/bottle, and the volume is 50 μl. Among them, LV-JW27+28 is combined immunization, and left and right leg muscles are injected with LV-JW27 and LV-JW28 respectively. A second immunization boost was performed one week later. Five weeks after the second immunization, the third immunization was performed. The third immunization boost was replaced with New Jersey VSV-G enveloped lentivirus. The dose was 5E+7 TU/mouse and the volume was 50 μL. Due to the lack of JW29 New Jersey VSV-G enveloped lentivirus, only four groups of LV-JW27, LV-JW28, LV-JW27+28, and LV-JW30 were given the third immunization boost.

### Effect of Therapeutic Lentivirus Vaccine on Hepatitis B Infection in Mice Changes of HBsAg Level in Peripheral Blood

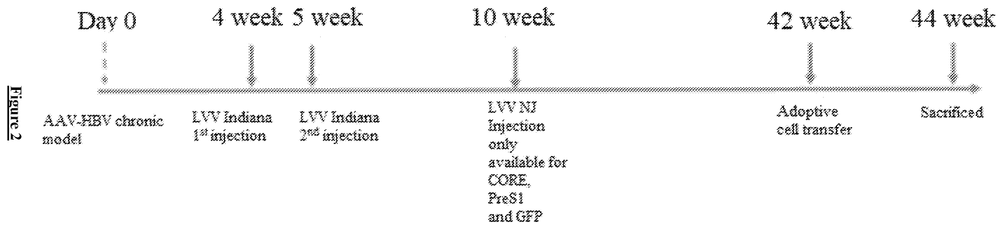

From the 4th week after the initial immunization, peripheral blood was collected weekly for detection of HBsAg and HBV DNA in the serum. The most obvious effect was the JW29 group (FIG. 3D). Five male mice showed a sharp decline in HBsAg after the initial immunization, and three female mice continued to decline in HBsAg after 9 weeks. By 19 weeks, there were 3 mice HBsAg turned negative, 5 HBsAg turned negative at 27 weeks, and 6 HBsAg turned negative at 38 weeks, with a negative conversion rate of 60% (Table 8). In the JW27 group (FIG. 3A), one male mouse died at the 7th week. After the first immunization, the HBsAg of the five male mice decreased and then rebounded. After 15 weeks, the HBsAg of 9 mice in the group continued to decline, and at 38 weeks, 4 of them achieved HBsAg negative (Table 8). In the JW28 group (FIG. 3B) from the first immunization to 15 weeks, HBsAg was in a fluctuating state and remained relatively stable. After 15 weeks, the HBsAg decreased to the end of the observation period. One HBsAg became negative (Table 8). The overall treatment no significant effect. In the JW27+JW28 combined immunization group (FIG. 3C), the HBsAg of the mice in the group remained stable from the first immunization to the third immunization in the 10th week. After the 10th week of immunization, the HBsAg of 4 mice began to decline continuously until they became negative. By the 27th week, 5 had achieved antigen negative, and by the end of the 38-week observation, a total of 6 had achieved HBsAg negative in the group (Table 8). The JW30 group was the control group (FIG. 3E). Except for one mouse to clear HBV by itself, the remaining 9 HBsAg remained stable during the observation period.

TABLE 8
HBsAg conversion

| | | HBsAg conversion | | | | | | | |
|---|---|---|---|---|---|---|---|---|---|
| Product# | Immunogens | 4 W | 8 W | 16 W | 19 W | 23 W | 27 W | 34 W | 38 W |
| JW27 | CORE | 0/10 | 0/9 | 0/9 | 0/9 | 1/9 | 2/9 | 3/9 | 4/9 |
| JW28 | PreS1 | 0/10 | 0/10 | 0/10 | 0/10 | 0/10 | 0/10 | 0/10 | 1/10 |
| JW27 + 28 | CORE + PreS1 | 0/10 | 0/10 | 0/10 | 0/10 | 1/10 | 5/10 | 5/10 | 6/10 |
| JW29 | large HBs | 0/10 | 0/10 | 1/10 | 3/10 | 4/10 | 4/10 | 5/10 | 6/10 |
| JW30 | GFP | 0/10 | 1/10 | 1/10 | 1/10 | 1/10 | 1/10 | 1/10 | 1/10 |

### Changes of HBV DNA Levels in Peripheral Blood

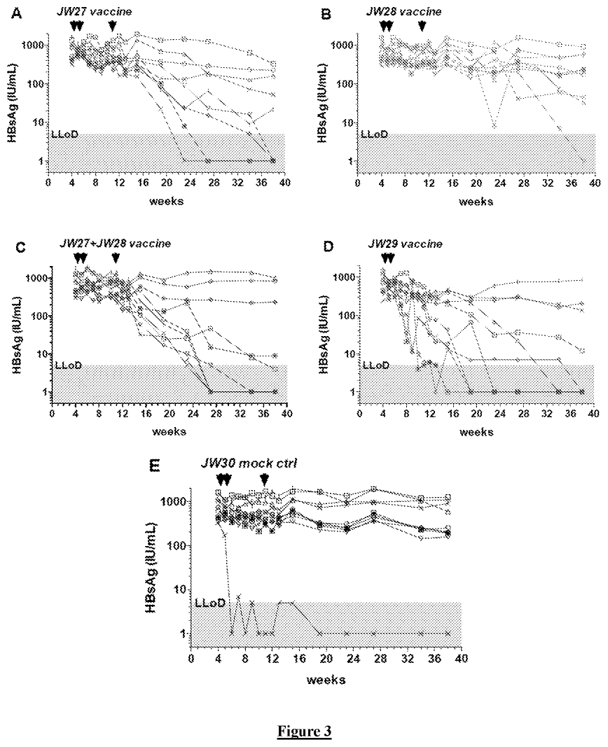

HBV DNA in the peripheral blood of mice in the JW27 group (FIG. 4A) gradually increased during the first immunization to 15 weeks and maintained a high level. Until 38 weeks, the HBV DNA of 4 male mice did not decrease significantly, but remained at a high level, while 5 of the female mice began to decline in HBV DNA after 15 weeks, and at 38 weeks, three female mice achieved HBV DNA clearance (Table 9). HBV DNA in the peripheral blood of mice in the JW28 group (FIG. 4B) also increased gradually from the first immunization to 15 weeks, and then maintained a high level overall. By 38 weeks, 1 mouse became negative (Table 9), and the remaining 9 remained Maintain a high level with no obvious effect. The HBV DNA in the peripheral blood of the JW27+JW28 combined immunization group (FIG. 4C) also maintained a stable or increased trend from the first immunization to 15 weeks. After 15 weeks, 8 HBV DNA decreased significantly, and 2 mice achieved HBV at 38 weeks DNA turns negative (Table 9). The peripheral blood HBV DNA of the mice in the JW29 group (FIG. 4D) fluctuated from the first immunization to 13 weeks and maintained a relatively stable level overall. After 15 weeks, the HBV DNA of 6 mice decreased significantly, and it was present at 23 weeks. 5 mice achieved HBV DNA negative, and a total of 6 mice HBV DNA turned negative by 38 weeks (Table 9), of which 4 female mice and 2 male mice, these 6 mice also achieved HBsAg negative, reaching Healed state. In the JW30 control group (FIG. 4E), with the exception of one mouse that became negative by itself, the remaining 9 HBV DNA remained stable during the observation period.

TABLE 9
HBV DNA clearance

| | | HBV DNA undetectable | | | | | | | |
|---|---|---|---|---|---|---|---|---|---|
| Product# | Immunogens | 4 W | 8 W | 16 W | 19 W | 23 W | 27 W | 34 W | 38 W |
| JW27 | CORE | 0/10 | 0/9 | 0/9 | | 1/9 | 2/9 | 2/9 | 3/9 |
| JW28 | PreS1 | 0/10 | 0/10 | 0/10 | | 0/10 | 0/10 | 0/10 | 1/10 |
| JW27 + 28 | CORE + PreS1 | 0/10 | 0/10 | 0/10 | | 2/10 | 2/10 | 1/10 | 2/10 |
| JW29 | large HBs | 0/10 | 0/10 | 1/10 | | 5/10 | 5/10 | 5/10 | 6/10 |
| JW30 | GFP | 0/10 | 0/10 | 0/10 | | 1/10 | 1/10 | 1/10 | 1/10 |

### Detection of HBV DNA to Eliminate Anti-HBs in Peripheral Blood

Surface antibody is a protective antibody produced by the body against hepatitis B virus. Its appearance indicates that it has specific immunity to HBV infection and the virus in serum has turned undetectable. Anti-HBs was detected in the peripheral blood serum of mice in the JW27, JW28, JW27+JW28, JW29, and JW30 groups at different time points, and the results are shown in the following table (Table 10): The time points of antibody conversion in the JW27, JW28 and JW29 groups All corresponded to the time point of HBsAg becoming negative.

TABLE 10
Anti-HBs antibody detection

| | | | anti-HBS (mIU/mL) | | | | | | |
|---|---|---|---|---|---|---|---|---|---|
| | | Mouse | 15 W | 19 W | 23 W | 27 W | 34 W | 38 W | 44 W |
| | | 233 | | | 0 | | | 0 | 0 |
| | | 237 | | | 0 | 84 | | 375 | 725 |
| | | 269 | | | 0 | | | 0 | 36 |
| | | 273 | | | 0 | | | 0 | 0 |
| 27 | CORE | 259 | | 0 | 262 | | | 659 | 933 |
| | | 179 | | | 0 | | | 0 | 0 |
| | | 216 | | | 0 | | | 0 | 0 |
| | | 182 | | | 0 | | | 0 | 0 |
| | | 220 | | | 0 | | | 0 | 0 |
| | | 266 | | | 0 | | | 0 | 0 |
| | | 263 | | | 0 | | | 0 | 0 |
| | | 264 | | | 0 | | | 0 | 0 |
| | | 225 | | | 0 | | | 95 | 511 |
| 28 | PreS1 | 224 | | | 0 | | | 0 | 0 |
| | | 196 | | | 0 | | | 0 | 0 |
| | | 188 | | | 0 | | | 0 | 0 |
| | | 199 | | | 0 | | | 0 | 0 |
| | | 190 | | 0 | 13 | | | 22 | 0 |
| | | 217 | | | 0 | | | 0 | 0 |
| | | 234 | | | 0 | 0 | | 591 | 696 |
| | | 265 | | | 0 | | | 0 | 0 |
| | | 246 | | | 0 | | | 0 | 0 |
| | | 236 | | | 14 | 4 | | 0 | 77 |
| 27 + 28 | CORE + | 270 | | | 0 | 167 | 17 | 168 | 358 |
| | PreS1 | 174 | 0 | 129 | 200 | | | 781 | 2772 |
| | | 201 | | 12 | 14 | | | 708 | 7245 |
| | | 187 | | | 0 | | 0 | 258 | 0 |
| | | 219 | | | 0 | 30 | | 42 | 37 |
| | | 210 | | | 0 | 0 | 0 | 10 | 0 |
| | | 247 | | | 21 | 1671 | | 1510 | |
| | | 249 | | | 0 | | | 0 | 0 |
| | | 232 | | | 0 | | | 0 | 0 |
| | | 241 | 0 | 11 | 108 | | | 987 | |
| 29 | Large | 268 | 0 | 132 | 829 | | | 1535 | 2556 |
| | S | 223 | | | 0 | | | 0 | 0 |
| | | 191 | | | 0 | | | 0 | 0 |
| | | 218 | | | 0 | 0 | 511 | 919 | |
| | | 203 | | | 0 | | | 0 | 0 |
| | | 211 | | | 0 | | | 0 | 0 |
| | | 239 | | | 0 | | | 0 | 0 |
| | | 227 | | | 0 | | | 0 | 0 |
| | | 226 | | | 0 | | | 0 | 0 |
| 30 | GFP | 260 | | | 0 | | | 0 | 0 |
| | | 243 | | | 0 | | | 0 | 0 |
| | | 183 | | | 0 | | | 0 | |
| | | 189 | | | 0 | | | 0 | |
| | | 221 | | | 0 | | | 0 | |
| | | 200 | | | 0 | | | 0 | |
| | | 181 | | | 0 | | | 0 | |

### Mice Spleen Cell IFN-γ ELISPOT Detection

The spleen cells of JW27, JW28, JW27+JW28, JW29 and JW30 groups were separated and added to the test wells for overnight culture, and the specific antigen genotype B preS1 polypeptide, genotype C preS1 polypeptide, preS2 polypeptide, Core polypeptide and HBsAg were added at the same time.

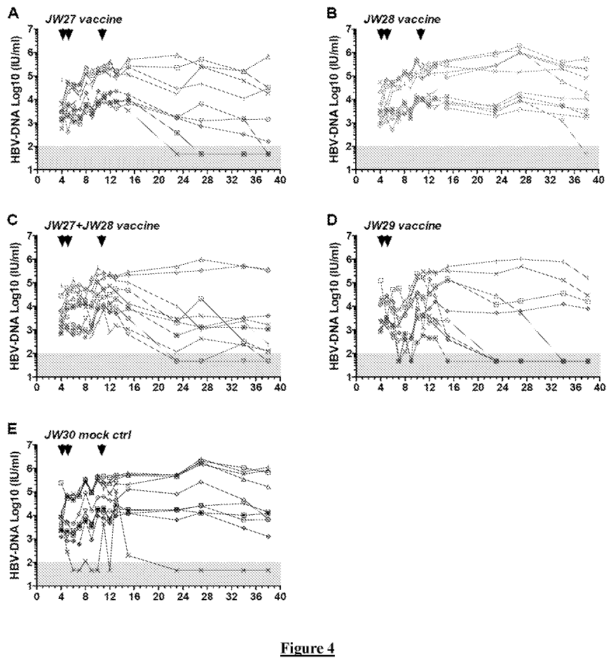

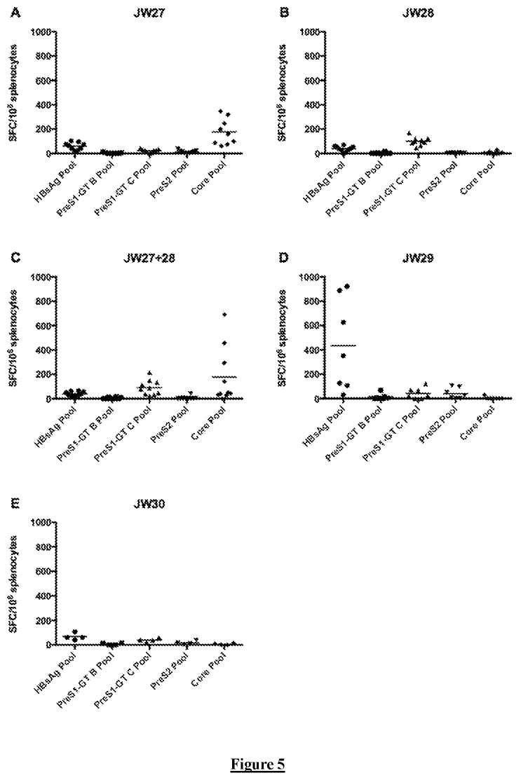

The results showed (FIG. 5) that JW27, JW28, JW27+28 and JW29 groups all had immune responses to specific antigens, with JW29 being the strongest.

### HBcAg Immunohistochemistry of Liver Tissue

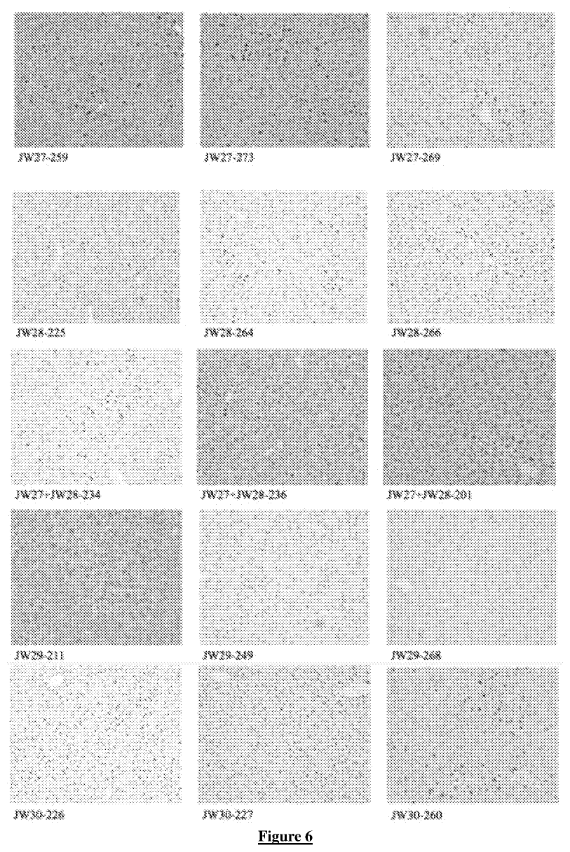

The representative liver tissue HBcAg immunohistochemical results of each group are shown in FIG. 6, the field of view under the microscope at 200×. Take 3 fields each to calculate the number of HBcAg positive cells and take the average value to get the positive rate. The results are shown in Table 11.

TABLE 11
IHC staining of HBcAg in liver cells

| Product | Mouse | HBcAg+ |
|---|---|---|
| JW27 | 233 | 10.01% |
| | 237 | 14.49% |
| | 269 | 9.38% |
| | 179 | 12.47% |
| | 182 | 25.05% |
| | 216 | 16.75% |
| | 220 | 15.57% |
| | 259 | 9.24% |
| | 273 | 13.80% |
| JW28 | 263 | 7.32% |
| | 264 | 8.68% |
| | 266 | 8.97% |
| | 188 | 19.00% |
| | 190 | 16.62% |
| | 196 | 12.55% |
| | 199 | 22.27% |
| | 219 | 7.06% |
| | 224 | 10.99% |
| | 225 | 5.58% |
| JW27 + 28 | 234 | 10.47% |
| | 246 | 9.38% |
| | 265 | 8.58% |
| | 174 | 13.73% |
| | 187 | 15.50% |
| | 201 | 10.00% |
| | 210 | 14.07% |
| | 219 | 13.17% |
| | 236 | 7.55% |
| | 270 | 11.70% |
| JW29 | 232 | 11.46% |
| | 249 | 0.46% |
| | 268 | 2.01% |
| | 191 | 10.29% |
| | 203 | 4.91% |
| | 211 | 1.54% |
| | 223 | 13.90% |
| | 241 | 9.19% |
| | 247 | 11.30% |
| JW30 | 227 | 13.09% |
| | 226 | 16.06% |
| | 239 | 14.75% |
| | 260 | 15.21% |

### 2.3 Conclusion

In summary, the hepatitis B therapeutic lentivirus vaccine of the present invention, especially the therapeutic vaccine capable of expressing LargeS antigen, can have a good hepatitis B therapeutic effect, and achieve HBsAg clearance and HBV DNA clearance.

1. A lentiviral vector for the treatment of hepatitis B, wherein the vector contains a nucleotide sequence encoding at least one hepatitis B virus antigen selected from the group consisting of core antigen (HBcAg), PreS1 antigen (PreS1) and Large S antigen (LargeS).

2. The lentiviral vector for treating hepatitis B according to claim 1, wherein the encoded hepatitis B virus antigen is a large S antigen (LargeS).

3. The lentiviral vector for treating hepatitis B according to claim 1, wherein the amino acid sequence of the core antigen (HBcAg) is the sequence set forth as SEQ ID NO:1; and/or the amino acid sequence of the PreS1 antigen (PreS1) is the sequence set forth as SEQ ID NO: 4 or as SEQ ID NO: 7; and/or the amino acid sequence of the large S antigen (LargeS) is the sequence set forth as SEQ ID NO: 10 or as SEQ ID NO: 13.

4. The lentiviral vector for treating hepatitis B according to claim 1, wherein the coding nucleotide sequence of the core antigen (HBcAg) is the sequence set forth as SEQ ID NO: 3; and/or the coding nucleotide sequence of the PreS1 antigen (PreS1) is the sequence set forth as SEQ ID NO: 6 or as SEQ ID NO: 9; and/or the coding nucleotide sequence of the Large S antigen (LargeS) is the sequence set forth as SEQ ID NO: 12 or as SEQ ID NO: 15.

5. A method for preparing lentiviral particles for treating hepatitis B, characterized in that the method comprises:
a) co-transfecting host cells with the lentiviral vector according to claim 1, a packaging vector expressing Gag, Rev and/or Pol protein, and an envelope vector expressing an envelope protein, or transfecting the lentiviral vector according to claim 1 into a host cell capable of expressing an envelope protein and one or more of Gag, Rev, and Pol proteins;
b) culturing the transfected host cell to package the lentiviral vector into lentiviral vector particles; and
c) harvesting the lentiviral vector particles produced in step b).

6. A preparation of lentiviral particles for the treatment of hepatitis B, wherein the lentiviral particles comprise the lentiviral vector according to claim 1.

7. A lentiviral vector according to claim 1, for the treatment and/or prevention of hepatitis B virus infection in a subject in need thereof, or for the treatment and/or prevention of diseases caused by hepatitis B virus infection.

8. The lentiviral vector or preparation of lentiviral particles for use according to claim 7, wherein the subject is a mammal.

9. A pharmaceutical composition for the treatment and/or prevention of hepatitis B virus infection or for the treatment and/or prevention of a disease caused by hepatitis B virus infection in a subject in need thereof, the pharmaceutical composition comprising the lentiviral vector according to claim 1, and a pharmaceutically acceptable carrier.

10. The pharmaceutical composition according to claim 9, wherein the subject is a mammal.
